(19) United States
(12) Patent Application Publication
(10) Pub. No.: US 2019/0170683 A1
(43) Pub. Date: Jun. 6, 2019

(54) THIN LAYER CHROMATOGRAPHY PLATE AND SAMPLE ANALYSIS METHOD USING SAME

(71) Applicant: Panasonic Intellecutal Property Management Co., Ltd., Osaka (JP)

(72) Inventors: TOSHIHIRO SAKAMOTO, Osaka (JP); CHIAKI SHIDO, Kyoto (JP)

(21) Appl. No.: 16/323,532

(22) PCT Filed: Dec. 13, 2017
(86) PCT No.: PCT/JP2017/044639

§ 371 Date: Feb. 6, 2019

(30) Foreign Application Priority Data
Dec. 26, 2016 (JP) 2016-250754
Dec. 26, 2016 (JP) 2016-250760

(51) Int. Cl. G01N 27/447; G01N 30/26; G01N 30/88; G01N 30/92; G01N 30/94; G01N 33/543

(52) U.S. Cl. CPC G01N 27/447; G01N 30/26; G01N 30/88; G01N 2030/885; G01N 30/94; G01N 33/543; G01N 30/92

(57) ABSTRACT

Provided is a thin layer chromatography plate that makes it possible to separate a plurality of components from each other more simply and more quickly. Thin layer chromatography plate includes substrate and separation layer that is disposed on substrate for separating multiple components included in a sample from each other. Separation layer has first layer that has a band shape and extends in first development direction and second layer that extends in second development direction orthogonal to first development direction. Second layer is in contact with first layer. First layer includes a hydrophilic porous body. Second layer includes a hydrophobic porous body.

### TECHNICAL FIELD

The present disclosure relates to a thin layer chromatography plate and a sample analysis using the same.

### BACKGROUND ART

Chromatography and electrophoresis, for example, have been known as a method for separating a specific component from a mixture containing multiple components. Thin layer chromatography which is a kind of chromatography techniques makes it possible to easily and quickly separate multiple components from each other.

As shown in FIG. 15, PTL 1 discloses thin layer chromatography plate 2000 provided with first separating agent layer 2031 and second separating agent layer 2032. Second separating agent layer 2032 is adjacent to first separating agent layer 2031. First separating agent layer 2031 and second separating agent layer 2032 are respectively formed from separating agents having different optical responses.

When thin layer chromatography plate 2000 is used, multiple components can be separated from each other as described below. Sample 2060 is placed on first separating agent layer 2031 and developed in direction X. Then, second separating agent layer 2032 is dried. Next, the orientation of thin layer chromatography plate 2000 is changed, and sample 2060 is developed in direction Y orthogonal to direction X. The multiple components are separated from each other in second separating agent layer 2032.

### CITATION LIST

### Patent Literature

PTL 1: WO 2011/149041 A

### SUMMARY OF THE INVENTION

According to the method disclosed in PTL 1, it is necessary that, after the sample is developed in first separating agent layer 2031, second separating agent layer 2032 is dried.

The present disclosure aims to provide a technique for separating multiple components from each other more easily and more quickly.

Specifically, the present disclosure provides a thin layer chromatography plate described below. The thin layer chromatography plate includes a substrate and a separation layer disposed on the substrate, the sepatatgion layer separating multiple components included in a sample from each other. The separation layer has a first layer that has a band shape and extends in a first development direction and a second layer that extends in a second development direction orthogonal to the first development direction. The second layer is in contact with the first layer, the first layer includes a hydrophilic porous body, and the second layer includes a hydrophobic porous body.

According to the thin layer chromatography plate in the present disclosure, multiple components can be separated from each other more easily and more quickly.

### BRIEF DESCRIPTION OF DRAWINGS

FIG. 1A is a plan view of a thin layer chromatography plate according to a first exemplary embodiment of the present disclosure.

FIG. 1B is a sectional view of the thin layer chromatography plate shown in FIG. 1A along second development direction Y.

FIG. 2A is a view showing a state where a sample is placed on the thin layer chromatography plate according to the first exemplary embodiment of the present disclosure.

FIG. 2B is a view showing a state where the thin layer chromatography plate shown in FIG. 2A is brought into contact with a first developing solvent.

FIG. 2C is a view showing a state where the thin layer chromatography plate shown in FIG. 2B is brought into contact with a second developing solvent by changing the orientation of the thin layer chromatography plate.

FIG. 3A is a plan view of a thin layer chromatography plate according to a second exemplary embodiment of the present disclosure.

FIG. 3B is a sectional view of the thin layer chromatography plate shown in FIG. 3A along second development direction Y.

FIG. 3C is a sectional view of a thin layer chromatography plate along second development direction Y according to a modification of the second exemplary embodiment of the present disclosure.

FIG. 3D is a sectional view of a thin layer chromatography plate along second development direction Y according to another modification of the second exemplary embodiment of the present disclosure.

FIG. 4A is a plan view of a thin layer chromatography plate according to a third exemplary embodiment of the present disclosure.

FIG. 4B is a sectional view of the thin layer chromatography plate shown in FIG. 4A along second development direction Y.

FIG. 5A is a plan view of a thin layer chromatography plate according to a fourth exemplary embodiment of the present disclosure.

FIG. 5B is a sectional view of the thin layer chromatography plate shown in FIG. 5A along line VB-VB.

FIG. 6A is a view showing a state where a sample is placed on the thin layer chromatography plate according to the fourth exemplary embodiment of the present disclosure.

FIG. 6B is a view showing a state where a voltage is applied to a pair of electrodes in the thin layer chromatography plate shown in FIG. 6A.

FIG. 6C is a view showing a state where the thin layer chromatography plate shown in FIG. 6B is brought into contact with a second developing solvent.

FIG. 7A is a plan view of a thin layer chromatography plate according to a fifth exemplary embodiment of the present disclosure.

FIG. 7B is a sectional view of the thin layer chromatography plate shown in FIG. 7A along second development direction Y.

FIG. 8A is a plan view of a thin layer chromatography plate according to a sixth exemplary embodiment of the present disclosure.

FIG. 8B is a sectional view of the thin layer chromatography plate shown in FIG. 8A along second development direction Y.

FIG. 9A is a view showing a state where a sample is placed on the thin layer chromatography plate according to the sixth exemplary embodiment of the present disclosure.

FIG. 9B is a view showing a state where the thin layer chromatography plate shown in FIG. 9A is brought into contact with a first developing solvent.

FIG. 9C is a view showing a state where the thin layer chromatography plate shown in FIG. 9B is brought into contact with a second developing solvent by changing the orientation of the thin layer chromatography plate.

FIG. 10A is a plan view of a thin layer chromatography plate according to a seventh exemplary embodiment of the present disclosure.

FIG. 10B is a sectional view of the thin layer chromatography plate shown in FIG. 10A along second development direction Y.

FIG. 10C is a sectional view of a thin layer chromatography plate along second development direction Y according to a modification of the seventh exemplary embodiment of the present disclosure.

FIG. 10D is a sectional view of a thin layer chromatography plate along second development direction Y according to another modification of the seventh exemplary embodiment of the present disclosure.

FIG. 11A is a plan view of a thin layer chromatography plate according to an eighth exemplary embodiment of the present disclosure.

FIG. 11B is a sectional view of the thin layer chromatography plate shown in FIG. 11A along second development direction Y.

FIG. 12A is a plan view of a thin layer chromatography plate according to a ninth exemplary embodiment of the present disclosure.

FIG. 12B is a sectional view of the thin layer chromatography plate shown in FIG. 12A along line XIIB-XIIB.

FIG. 13A is a view showing a state where a sample is placed on the thin layer chromatography plate according to the ninth exemplary embodiment of the present disclosure.

FIG. 13B is a view showing a state where a voltage is applied to a pair of electrodes in the thin layer chromatography plate shown in FIG. 13A.

FIG. 13C is a view showing a state where the thin layer chromatography plate shown in FIG. 13B is brought into contact with a second developing solvent.

FIG. 14A is a plan view of a thin layer chromatography plate according to a tenth exemplary embodiment of the present disclosure.

FIG. 14B is a sectional view of the thin layer chromatography plate shown in FIG. 14A along second development direction Y.

FIG. 14C is a sectional view of a thin layer chromatography plate along second development direction Y according to a modification of the tenth exemplary embodiment of the present disclosure.

FIG. 14D is a sectional view of a thin layer chromatography plate along second development direction Y according to another modification of the tenth exemplary embodiment of the present disclosure.

FIG. 15 is a plan view of a conventional thin layer chromatography plate.

### DESCRIPTION OF EMBODIMENTS

### Underlying Knowledge of the Present Disclosure

A human skin state can be checked by analyzing proteins included in skin. The protein analysis is conducted in the manner described below, for example. A sample such as a surface skin is extracted from the skin of a subject. The sample includes a plural kinds of proteins. The multiple proteins included in the sample are separated from each other using thin layer chromatography. Each of the separated proteins is identified.

For example, if the sample includes a protein related to by rough skin, it is found that the subject has rough skin If the skin state of the subject can be recognized, cosmetics suitable for the subject can be recommended. It is convenient to check the skin state of the subject and recommend cosmetics based on the check result in cosmetics retail stores. When doing so, protein analysis needs to be quickly conducted during a waiting time of the subject.

A thin layer chromatography plate according to a first aspect of the present disclosure has the configurations described below.

Specifically, the thin layer chromatography plate includes: a substrate; and a separation layer disposed on the substrate for separating multiple components included in a sample from each other. The separation layer has a first layer that has a band shape and extends in a first development direction and a second layer that extends in a second development direction orthogonal to the first development direction. The second layer is in contact with the first layer, the first layer includes a hydrophilic porous body, and the second layer includes a hydrophobic porous body.

According to the first aspect, the second layer in the separation layer includes a hydrophobic porous body, whereby water is difficult to penetrate into the second layer. That is, if a developing solvent is selected as appropriate, pores in the porous body constituting the second layer hardly contains the developing solvent, after the multiple components included in the sample are developed in the first development direction. Therefore, it is unnecessary to dry the second layer after the multiple components are developed in the first development direction. Thus, the multiple components can be separated from each other more easily and more quickly.

According to a second aspect of the present disclosure, the first layer and the second layer of the thin layer chromatography plate according to the first aspect are both disposed on the substrate, and a lateral surface of the first layer is in contact with a lateral surface of the second layer, for example. According to the second aspect, the developing solvent can be easily moved from the first layer to the second layer.

According to a third aspect of the present disclosure, the separation layer of the thin layer chromatography plate according to the first aspect further includes a third layer that is in contact with the second layer, and the first layer, the second layer, and the third layer are arrayed in sequence in the second development direction, for example. In this configuration, the third layer includes a porous body. Further, at least one requirement selected from among a requirement of a composition of the third layer being different from a composition of the second layer and a requirement of a structure of the third layer being different from a structure of the second layer is satisfied.

According to the third aspect, the second layer in the separation layer includes a hydrophobic porous body, whereby water is difficult to penetrate into the second layer. That is, if a developing solvent is selected as appropriate, pores in the porous body constituting the second layer hardly contains the developing solvent, after the multiple components included in the sample are developed in the first development direction. Therefore, it is unnecessary to dry the second layer after the multiple components are developed in the first development direction. Thus, the multiple components can be separated from each other more easily and more quickly. The third layer in the separation layer induces an interaction different from the interaction induced by the second layer, with respect to the multiple components included in the sample. Therefore, the multiple components which are not separated from each other in the second layer are separated from each other in the third layer.

According to a fourth aspect of the present disclosure, the first layer, the second layer, and the third layer of the thin layer chromatography plate according to the third aspect are disposed on the substrate, and a lateral surface of the first layer and a lateral surface of the second layer are in contact with each other, for example. According to the fourth aspect, the developing solvent can be easily moved from the first layer to the second layer.

According to a fifth aspect of the present disclosure, the separation layer of the thin layer chromatography plate according to the second aspect or the fourth aspect further includes a functional layer that extends in the second development direction and includes the hydrophobic porous body or another hydrophobic porous body, for example. The functional layer is in contact with the first layer, and the functional layer, the first layer, and the second layer are arrayed in sequence in the second development direction. According to the fifth aspect, a gradient occurring in a movement distance of the developing solvent in the second development direction is reduced in the functional layer. Thus, the multiple components can move straight in the second development direction in the second layer.

According to a sixth aspect of the present disclosure, the second layer of the thin layer chromatography plate according to the first aspect or the third aspect is disposed on the substrate, the first layer is disposed on the second layer, and a lower surface of the first layer and an upper surface of the second layer are in contact with each other, for example. According to the sixth aspect, a gradient occurring in a movement distance of the developing solvent in the second development direction is reduced in a part of the second layer. Thus, the multiple components can move straight in the second development direction in the second layer.

According to the seventh aspect of the present disclosure, the first layer of the thin layer chromatography plate according to the sixth aspect is located between one end and the other end of the second layer in the second development direction, for example. According to the seventh aspect, the multiple components can move straight in the second development direction in the second layer.

According to an eighth aspect of the present disclosure, the hydrophobic porous body of the thin layer chromatography plate according to any one of the first to seventh aspects is an aggregate of silica gel particles modified with a hydrophobic functional group, for example. According to the eighth aspect, it is unnecessary to dry the second layer after the multiple components included in the sample are developed in the first development direction. Thus, the multiple components can be separated from each other more easily and more quickly.

According to a ninth aspect of the present disclosure, the thin layer chromatography plate according to any one of the first to eighth aspects further includes a pair of electrodes disposed at both ends of the first layer in the first development direction, for example. According to the ninth aspect, electrophoresis of the multiple components included in the sample can be achieved.

A sample analysis method according to a tenth aspect of the present disclosure includes the following steps. Specifically, the sample analysis method includes: placing a sample onto a first layer of the thin layer chromatography plate according to any one of the first to eighth aspects; and bringing an end of the first layer in the first development direction into contact with a first developing solvent. The sample analysis method further includes changing the orientation of the thin layer chromatography plate to bring the thin layer chromatography plate into contact with a second developing solvent containing an organic solvent with the first layer being impregnated with the first developing solvent.

According to the tenth aspect, due to the second layer in the separation layer including the hydrophobic porous body, pores in the porous body constituting the second layer hardly contains the first developing solvent, after the multiple components included in the sample are developed in the first development direction. Therefore, it is unnecessary to dry the second layer after the multiple components included in the sample are developed in the first development direction. Thus, the multiple components can be separated from each other more easily and more quickly.

According to an eleventh aspect of the present disclosure, the first developing solvent used in the sample analysis method according to the tenth aspect is an aqueous solution, for example. According to the eleventh aspect, it is unnecessary to dry the second layer after the multiple components included in the sample are developed in the first development direction. Thus, the multiple components can be separated from each other more easily and more quickly.

According to a twelfth aspect of the present disclosure, the second developing solvent used in the sample analysis method according to the tenth or eleventh aspect is a mixed solvent containing the organic solvent and water, for example. According to the tenth aspect, the multiple components included in the sample can be easily dissolved in the second developing solvent.

A sample analysis method according to a thirteenth aspect of the present disclosure includes the following steps. Specifically, the sample analysis method includes: placing a sample onto the first layer of the thin layer chromatography plate according to the ninth aspect; applying a voltage to the pair of electrodes; and bringing the thin layer chromatography plate into contact with a developing solvent containing an organic solvent.

According to the eleventh aspect, due to the second layer in the separation layer including the hydrophobic porous body, pores in the porous body constituting the second layer hardly contains the developing solvent, after the multiple components included in the sample are developed in the first development direction. Therefore, it is unnecessary to dry the second layer after the multiple components included in the sample are developed in the first development direction. Thus, the multiple components can be separated from each other more easily and more quickly.

Exemplary embodiments of the present disclosure will be described below with reference to the drawings. The present disclosure is not limited to the following exemplary embodiments.

### First Exemplary Embodiment

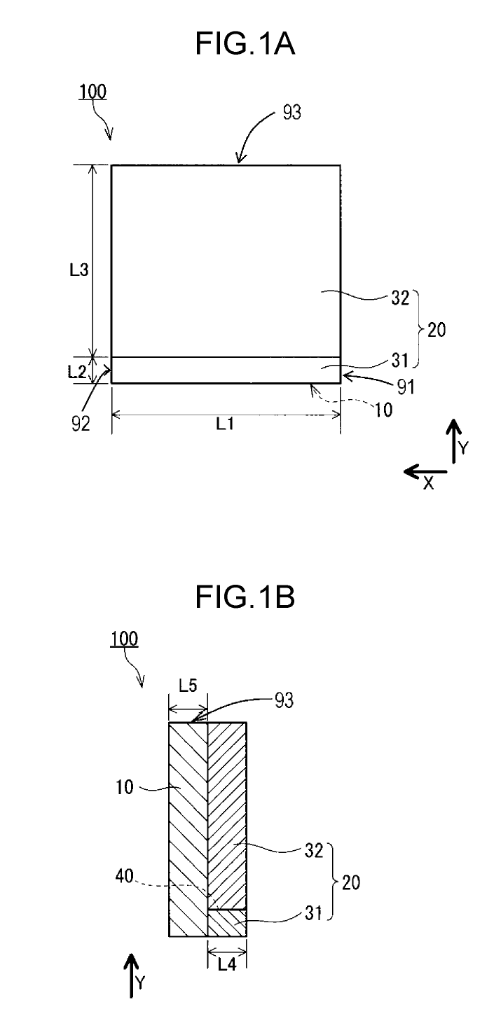

As shown in FIGS. 1A and 1B, thin layer chromatography plate 100 (hereinafter referred to as “TLC plate 100”) according to the present first exemplary embodiment has substrate 10 and separation layer 20. Substrate 10 has a plate shape, for example. Substrate 10 has, for example, a rectangular shape in a plan view. Separation layer 20 is disposed on substrate 10. Separation layer 20 covers the surface of substrate 10. Substrate 10 has two pairs of end faces facing each other. In the present exemplary embodiment, first development direction X extends from one end face of one of two pairs of end faces of substrate 10 to the other end face. Multiple components included in the sample are developed in first development direction X in a first stage. Second development direction Y extends from one end face of the other of two pairs of end faces of substrate 10 to the other end face. Multiple components included in the sample are developed in second development direction Y in a second stage. Second development direction Y is orthogonal to first development direction X.

Separation layer 20 separates the multiple components included in the sample from each other. Separation layer 20 includes first layer 31 and second layer 32. First layer 31 is a band-shaped layer. First layer 31 has a rectangular band shape in a plan view. First layer 31 extends in first development direction X. First layer 31 extends from one (end face 91) of a pair of end faces (end face 91 and end face 92) of substrate 10 to the other (end face 92) in first development direction X. Note that first layer 31 may not extend to the other end face (end face 92) of the pair of end faces of substrate 10 in first development direction X.

Second layer 32 has a rectangular shape in a plan view. Second layer 32 extends in second development direction Y. Second layer 32 is in contact with first layer 31. When separation layer 20 is viewed in a plan view, one side (long side) of first layer 31 is in contact with one side of second layer 32. The length of one side (long side) of first layer 31 is equal to the length of one side of second layer 32. First layer 31 and second layer 32 constitute boundary face 40. Boundary face 40 extends in first development direction X. Second layer 32 extends from boundary face 40 to end face 93 of substrate 10 in second development direction Y. Note that second layer 32 may not extend to end face 93 of substrate 10 in second development direction Y.

In the present exemplary embodiment, first layer 31 and second layer 32 are both disposed on substrate 10. In other words, first layer 31 and second layer 32 are both in contact with substrate 10. A lateral surface of first layer 31 and a lateral surface of second layer 32 are in contact with each other. When the multiple components are developed in second development direction Y, a developing solvent can easily move from first layer 31 to second layer 32 through boundary face 40.

The material of substrate 10 is not particularly limited, as long as it can maintain the shape of TLC plate 100 without being eluted in a developing solvent. The material of substrate 10 is glass, resin, metal, or paper, for example. Substrate 10 is typically a glass plate or an aluminum film.

First layer 31 includes a hydrophilic porous body. In the present specification, “being hydrophilic” means that, when water is brought into contact with the porous body, water can move in the porous body at a rate of 5 mm/min or higher due to capillary force, for example. When water is selected as the developing solvent, first layer 31 can carry water from one end to the other end of first layer 31 in first development direction X due to capillary force. The hydrophilic porous body is not particularly limited. The material of the hydrophilic porous body includes at least one selected from the group consisting of a fiber material, and an inorganic material and a polymer material which are hydrophilic, for example.

The fiber material includes at least one selected from the group consisting of a plant fiber, an animal fiber, a recycled fiber, a synthetic fiber, and a glass fiber, for example. The plant fiber includes cellulose, for example. The synthetic fiber includes cellulose acetate, for example.

The hydrophilic polymer material includes at least one selected from the group consisting of agarose, dextran, and mannan, for example. The inorganic material includes at least one selected from the group consisting of alumina, silicon dioxide, and zirconia, for example.

The hydrophilic porous body is filter paper, for example. The hydrophilic porous body is an aggregate of at least one kind of inorganic particles selected from the group consisting of alumina particles, silica gel particles, silicon pillar, zeolite particles, diatomaceous earth, and zirconia particles, for example.

An average pore diameter of first layer 31 may be within a range from 0.01 μm to 100 μm. When the hydrophilic porous body is an aggregate of inorganic particles, an average particle diameter of the inorganic particles may be within a range from 1 μm to 100 μm. The “average pore diameter” can be measured with the following method. Specifically, the surface or cross-section of first layer 31 is observed with an electron microscope (for example, an scanning electron microscope). Pore diameters of a plurality of observed pores (for example, random 50 pores) are measured. The average pore diameter is determined based on the average value calculated using the measured values. The diameter of a circle having an area equal to the area of the pore observed with the electron microscope can be regarded as the pore diameter. The “average particle diameter” can be measured with the following method. Specifically, the surface or cross-section of first layer 31 is observed with an electron microscope, and diameters of random number (for example, 50) of particles constituting first layer 31 are measured. The average particle diameter is determined based on the average value calculated using the obtained measured values. The diameter of a circle having an area equal to the area of the particle observed with the electron microscope can be regarded as the particle diameter.

First layer 31 may further include an additive. Examples of the additive include a fluorescence indicator, a binder, and a metal oxide.

Examples of the fluorescence indicator include magnesium tungstate and zinc silicate containing manganese. When first layer 31 includes the fluorescence indicator, positions of the multiple components can be detected by irradiating first layer 31 with ultraviolet ray.

The binder includes at least one selected from the group consisting of an inorganic binder, an organic fiber, a thickener, and an organic binder, for example. Examples of the inorganic binder include plaster and colloidal silica. Examples of the organic fiber include microfibrillar cellulose. Examples of the thickener include hydroxyethyl cellulose and carboxymethyl cellulose. Examples of the organic binder include polyvinyl alcohol and polyacrylic acid. When first layer 31 includes the binder, adhesiveness between substrate 10 and first layer 31 is improved. When the porous body of first layer 31 is an aggregate of inorganic particles, durability of the aggregate of inorganic particles is improved due to the binder.

The metal oxide includes at least one selected from the group consisting of titanium oxide, aluminum oxide, tin oxide, zinc oxide, tungsten oxide, manganese oxide, nickel oxide, copper oxide, and magnesium oxide, for example. The metal oxide may be charged when getting wet with the developing solvent. Therefore, when first layer 31 includes the metal oxide, the interaction between the multiple components included in the sample and first layer 31 varies. Thus, the multiple components may be easily separated from each other in first layer 31.

The above-mentioned additives may be mixed into the porous body constituting first layer 31. When the additive is a binder or a metal oxide, the additive may cover the surface of the porous body. The additive may coat the surfaces of inorganic particles constituting the porous body.

Second layer 32 includes a hydrophobic porous body. In the present specification, “being hydrophobic” means that, when water is brought into contact with the porous body, water can move in the porous body at a rate lower than 5 mm/min due to capillary force, or water does not move inside the porous body due to capillary force, for example. When water moves inside the hydrophobic porous body, the movement rate of water may be lower than 1 mm/min. When second layer 32 is brought into contact with water, water hardly penetrates into second layer 32. When the developing solvent contains an organic solvent, second layer 32 can carry the developing solvent from one end to the other end of second layer 32 in second development direction Y due to capillary force. The hydrophobic porous body is not particularly limited. The material of the hydrophobic porous body is, for example, a hydrophobic polymer material. The hydrophobic polymer material includes at least one selected from the group consisting of fluororesin, polystyrene, polyethylene, and polypropylene, for example.

The hydrophobic porous body is, for example, a porous body of a hydrophobic polymer material or an aggregate of hydrophobic polymer material particles. The hydrophobic porous body is an aggregate of inorganic particles modified with a hydrophobic functional group, for example. The hydrophobic functional group includes a functional group having a hydrocarbon group at the end, for example. The hydrocarbon group includes at least one selected from the group consisting of an octadecyl group, an octyl group, a t-butyl group, a trimethylsilyl group, and a phenyl group, for example. The inorganic particles include at least one kind selected from the group consisting of alumina particles, silica gel particles, silicon pillar, zeolite particles, diatom earth, and zirconia particles, for example. The hydrophobic porous body is typically an aggregate of silica gel particles modified with a hydrophobic functional group. Whether the inorganic particles are modified with a hydrophobic functional group can be confirmed by conducting an elemental analysis on the cross-section of second layer 32, for example. The elemental analysis can be conducted by X-ray photoelectron spectroscopy (XPS) or energy dispersive X-ray spectroscopy (EDX), for example.

An average pore diameter of second layer 32 may be within a range from 0.01 μm to 100 μm. When the hydrophobic porous body is an aggregate of inorganic particles modified with a hydrophobic functional group, an average particle diameter of the inorganic particles may be within a range from 1 μm to 100 μm. Second layer 32 may further include any of the above-mentioned additives.

Length L1 of first layer 31 in first development direction X is not particularly limited. Length L1 is determined according to the porous body constituting first layer 31, a size of a container for housing TLC plate 100, and the like. Length L1 is 10 mm to 100 mm, for example. The length of second layer 32 and the length of substrate 10 in first development direction X are typically equal to length L1.

Length L2 of first layer 31 in second development direction Y is not particularly limited. Length L2 is determined according to an amount of the sample to be placed on first layer 31, for example. As length L2 is smaller, the multiple components are more easily separated from each other when the multiple components are developed in second development direction Y. Length L2 is 0.5 mm to 10 mm, for example.

Length L3 of second layer 32 in second development direction Y is not particularly limited. Length L3 is determined according to the porous body constituting second layer 32, a size of a container for housing TLC plate 100, and the like. Length L3 is 20 mm to 200 mm, for example. The length of substrate 10 in second development direction Y is typically equal to the total of length L2 and length L3.

Thickness L4 of first layer 31 is not particularly limited. Thickness L4 is determined according to the porous body constituting first layer 31, for example. Thickness L4 is 0.05 mm to 1 mm, for example. The thickness of second layer 32 is typically equal to thickness L4 of first layer 31.

Thickness L5 of substrate 10 is not particularly limited as long as the shape of TLC plate 100 can be maintained. Thickness L5 is 0.1 mm to 5 mm, for example.

Next, a manufacturing method of TLC plate 100 will be described.

First, a first dispersion liquid containing inorganic particles is prepared. The first dispersion liquid can be obtained by dispersing inorganic particles into a coating solvent.

The coating solvent includes at least one selected from the group consisting of water and an organic solvent, for example. The organic solvent includes at least one selected from the group consisting of alcohol, ketone, ether, nitrile, sulfoxide, sulfone, ester, carboxylic acid, amide, hydrocarbon, aromatic hydrocarbon, and halogen-containing compound, for example.

Examples of alcohol include methanol, ethanol, and isopropyl alcohol. Examples of ketone include acetone and ethyl methyl ketone. Examples of ether include tetrahydrofuran and dioxane. Examples of nitrile include acetonitrile. Examples of sulfoxide include dimethyl sulfoxide. Examples of sulfone include sulfolane. Examples of ester include ethyl acetate. Examples of carboxylic acid includes formic acid and acetic acid. Examples of amide include dimethylformamide. Examples of hydrocarbon include pentane and hexane. Examples of aromatic hydrocarbon include benzene, toluene, and xylene. Examples of halogen-containing compound include methylene chloride, chloroform, bromoform, chlorobenzene, and bromobenzene.

The first dispersion liquid is applied on a part of the surface of substrate 10 to form a coating film. The coating film is dried, whereby first layer 31 is formed on substrate 10. When the hydrophilic porous body is filter paper, first layer 31 is formed on substrate 10 by bonding the hydrophilic porous body to a part of the surface of substrate 10 under pressure.

Next, a second dispersion liquid containing inorganic particles modified with a hydrophobic functional group is prepared. The second dispersion liquid can be obtained by dispersing inorganic particles modified with a hydrophobic functional group into a coating solvent. The materials mentioned above can be used for the coating solvent.

The second dispersion liquid is applied on a part of the surface of substrate 10 to form a coating film. The coating film is dried, whereby second layer 32 is formed on substrate 10. When the hydrophobic porous body is a porous body of a hydrophobic polymer material, second layer 32 is formed on substrate 10 by bonding the hydrophobic porous body to a part of the surface of substrate 10 under pressure.

The second dispersion liquid may contain inorganic particles not modified with a hydrophobic functional group, in place of inorganic particles modified with a hydrophobic functional group. In such a case, second layer 32 is formed in the manner described below. The second dispersion liquid is applied on substrate 10 to form a coating film. The coating film is dried, whereby a untreated layer of second layer 32 is formed. A silane coupling agent having a hydrophobic functional group is applied on the untreated layer. The silane coupling agent is reacted with the inorganic particles included in the untreated layer. Thus, second layer 32 is formed on substrate 10. The silane coupling agent may be applied on a coating film, not on the untreated layer.

The silane coupling agent is not particularly limited. The silane coupling agent may include at least one selected from the group consisting of dimethyloctadecylchlorosilane, dimethyloctylchlorosilane, t-butyldimethylchlorosilane, trimethylchlorosilane, and phenyldimethylchlorosilane.

First layer 31 and second layer 32 may be formed by the following method. The first dispersion liquid is applied on the entire surface of substrate 10 to form a coating film The coating film is dried, whereby a untreated layer of second layer 32 and first layer 31 are formed on substrate 10. A silane coupling agent having a hydrophobic functional group is applied on the untreated layer of second layer 32. The silane coupling agent is reacted with the inorganic particles. Thus, first layer 31 and second layer 32 are formed on substrate 10.

The order of formation of first layer 31 and second layer 32 on substrate 10 is not particularly limited. First layer 31 may be formed on substrate 10 after second layer 32 is formed on substrate 10.

Next, the sample analysis method using TLC plate 100 will be described.

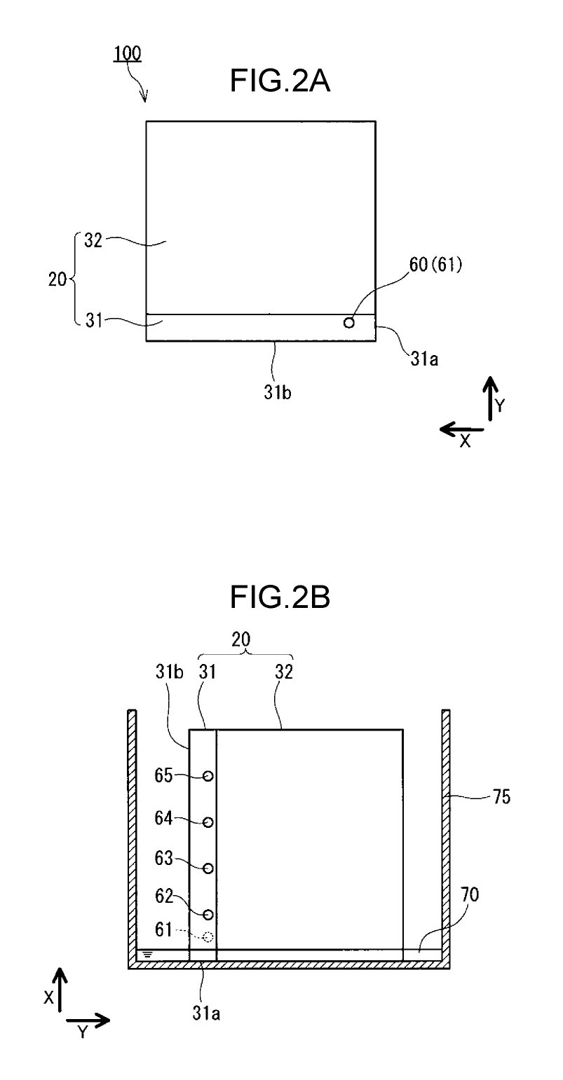

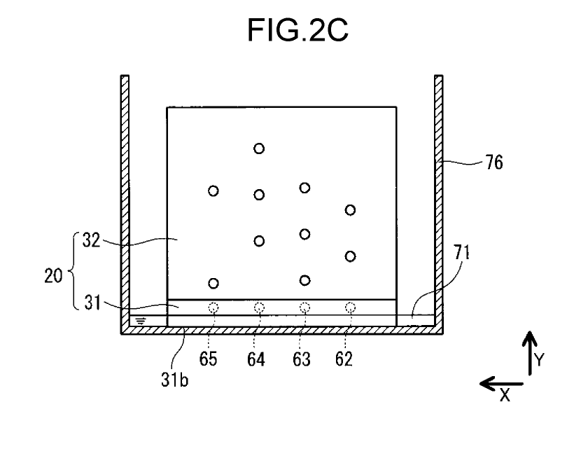

First, sample 60 is placed on first layer 31 of separation layer 20 of TLC plate 100, as shown in FIG. 2A. When sample 60 is placed on first layer 31, sample 60 penetrates into first layer 31, so that circular spot 61 is formed. Sample 60 is an aqueous solution containing a plurality of proteins, for example. The content of the plurality of proteins in sample 60 is from 0.01 wt. % to 1 wt. %, for example. The volume of sample 60 placed on first layer 31 is 0.1 μL to 2 μL, for example. The position where sample 60 is to be placed on first layer 31 is not particularly limited, as long as sample 60 is not in direct contact with the first developing solvent and the second developing solvent.

Then, as shown in FIG. 2B, TLC plate 100 is placed in container 75 with end 31a of first layer 31 in first development direction X being directed downward. Container 75 contains first developing solvent 70. Container 75 is a glass jar, for example. Container 75 may be installed inside an analyzing device (not shown).

First developing solvent 70 is not particularly limited, as long as it does not move to the inside of second layer 32 when being brought into contact with the surface of second layer 32. First developing solvent 70 is water or an aqueous solution, for example. A solute of the aqueous solution contains at least one selected from the group consisting of phosphate, citrate, acetate, and borate, for example. The aqueous solution may be a buffer solution such as a phosphate buffer solution, a tris buffer solution, a citrate buffer solution, an acetate buffer solution, or a borate buffer solution. In the present exemplary embodiment, first developing solvent 70 does not contain an organic solvent. However, first developing solvent 70 may contain an organic solvent. When first developing solvent 70 contains an organic solvent, first developing solvent 70 typically contains 80 volume % or more water.

When TLC plate 100 is placed in container 75, end 31a of first layer 31 is in contact with first developing solvent 70. The liquid level of first developing solvent 70 is set to prevent direct contact between first developing solvent 70 and sample 60. Due to the capillary force, first developing solvent 70 moves in first development direction X from end 31a of first layer 31. When first developing solvent 70 and sample 60 are brought into contact with each other, the multiple components included in sample 60 are dissolved into first developing solvent 70. The multiple components dissolved in first developing solvent 70 move in first development direction X along with first developing solvent 70. The multiple components move while repeatedly adsorbing and desorbing to and from the porous body constituting first layer 31. The frequency of adsorption and desorption varies in each component, and thus, the multiple components are separated from each other in first layer 31. Due to the development of sample 60 in first development direction X, spots 62, 63, 64, and 65 are newly generated. Spots 62, 63, 64, and 65 respectively indicate that any of the multiple components included in the sample is located therein.

Then, the orientation of TLC plate 100 is changed. The analyzing device may include a mechanism for changing the orientation of TLC plate 100. As shown in FIG. 2C, TLC plate 100 is placed in container 76 with end 31b of first layer 31 in second development direction Y being directed downward. Container 76 contains second developing solvent 71. Container 76 is a glass jar, for example. Container 76 may be installed inside the analyzing device.

Second developing solvent 71 is not particularly limited, as long as it contains an organic solvent. Second developing solvent 71 contains an organic solvent, so that it can penetrate into second layer 32. The materials mentioned above as examples of the coating solvent can be used as the organic solvent. The organic solvent contains at least one selected from the group consisting of methanol, ethanol, isopropyl alcohol, acetonitrile, and acetic acid, for example. When second developing solvent 71 contains carboxylic acid and the sample contains proteins, the frequency of absorption and desorption of proteins to and from the porous body constituting second layer 32 is improved. Second developing solvent 71 may contain the organic solvent in an amount of 20 wt. % or more. Second developing solvent 71 may contain water in addition to the organic solvent. That is, second developing solvent 71 may be a mixed solvent containing the organic solvent and water. When second developing solvent 71 contains water and the sample contains proteins, solubility of the proteins in second developing solvent 71 is improved. In other words, the multiple components included in the sample can be easily dissolved in second developing solvent 71. Specific examples of second developing solvent 71 include a mixed solvent in which isopropyl alcohol, acetic acid, and water are mixed in a weight ratio of 40:5:55.

When TLC plate 100 is placed in container 76, end 31b of first layer 31 is in contact with second developing solvent 71. At that time, first layer 31 is impregnated with first developing solvent 70. The liquid level of second developing solvent 71 is set to prevent direct contact between second developing solvent 71 and spots 62, 63, 64, and 65. Second developing solvent 71 penetrates into first layer 31. Second developing solvent 71 moves from end 31b of first layer 31 in second development direction Y due to capillary force, along with first developing solvent 70 penetrating into first layer 31. When second developing solvent 71 is brought into contact with the multiple components located in spots 62, 63, 64, and 65, the multiple components are dissolved into second developing solvent 71. The multiple components dissolved in second developing solvent 71 move in second development direction Y along with second developing solvent 71. The multiple components move while repeatedly adsorbing and desorbing to and from the porous body constituting second layer 32. The multiple components which are not separated from each other in first layer 31 are separated from each other in second layer 32.

Second layer 32 of TLC plate 100 includes a hydrophobic porous body. First developing solvent 70 hardly penetrates into second layer 32. That is, pores in the porous body constituting second layer 32 hardly contain first developing solvent 70, after the multiple components included in sample 60 are developed by first developing solvent 70. Therefore, it is unnecessary to dry second layer 32 after the multiple components included in the sample are developed in first development direction X. TLC plate 100 can be brought into contact with second developing solvent 71 with first layer 31 being impregnated with first developing solvent 70. In other words, it is unnecessary to heat TLC plate 100 to a temperature higher than room temperature or leave TLC plate 100 in an atmosphere with a pressure lower than atmospheric pressure, during a period from when TLC plate 100 is lifted up from first developing solvent 70 till TCL plate 100 is brought into contact with second developing solvent 71. According to TLC plate 100, the multiple components can be developed in second development direction Y just after the development of the multiple components in first development direction X. Thus, the multiple components can be separated from each other more easily and more quickly. Note that, with the sample analysis method in the present exemplary embodiment, separation layer 20 may be dried before TLC plate 100 is brought into contact with second developing solvent 71.

A method for detecting positions of multiple components is not particularly limited, and any known methods can be employed. For example, when first layer 31 and second layer 32 contain a fluorescence indicator, separation layer 20 may be irradiated with ultraviolet ray to detect the positions of multiple components. In such a case, each of the multiple components can be a compound that absorbs ultraviolet ray. The analyzing device may have a mechanism for emitting ultraviolet ray. The positions of the multiple components may be detected by depositing a coloring reagent onto separation layer 20. In such a case, TLC plate 100 may be heated as necessary. Any known coloring reagent can be used. Examples of the coloring reagent include anisaldehyde, phosphomolybdic acid, iodine, ninhydrin, chameleon solution, 2,4-dinitrophenylhydrazine, manganese chloride, and bromocresol green.

Under the same condition, the positions of the multiple components after sample 60 is developed are determined for each component. Therefore, with the sample analysis method according to the present exemplary embodiment, each of the separated multiple components can be identified. For example, a component having a known structure is developed on TLC plate 100 under the condition same as the condition for developing sample 60. Data in which the position of the component after the development and the structure of the component are associated with each other is acquired. This data may be stored in a memory of the analyzing device in advance. Through comparison with the data, each of the multiple components can be identified based on the position of each component after sample 60 is developed.

### Second Exemplary Embodiment

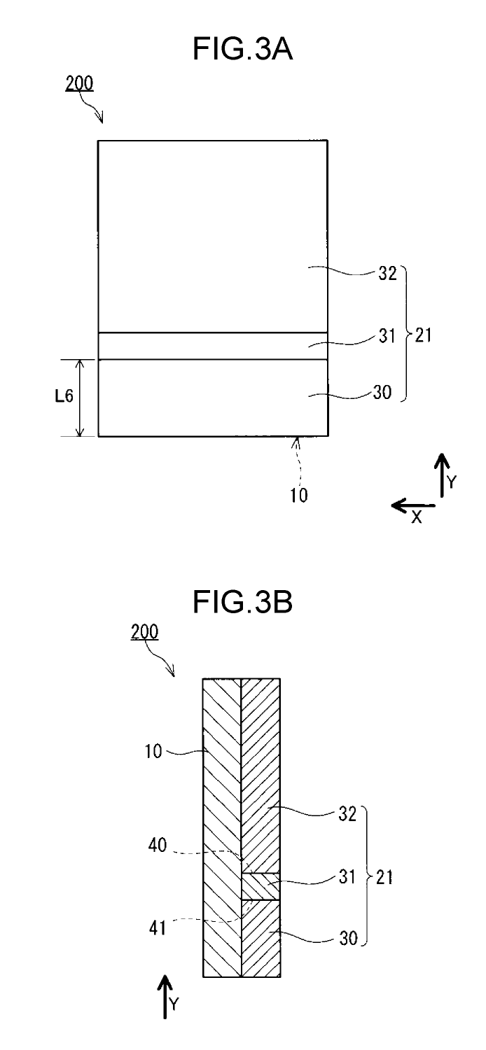

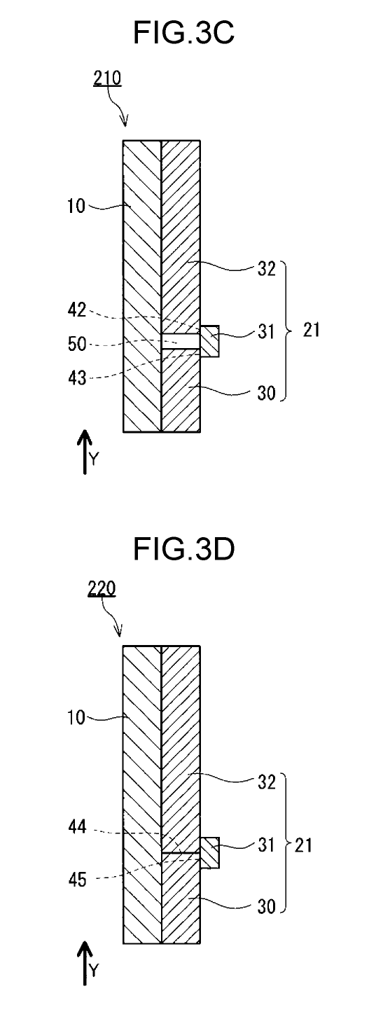

As shown in FIGS. 3A and 3B, TLC plate 200 according to the present second exemplary embodiment includes separation layer 21 having first layer 31, second layer 32, and functional layer 30. A structure of TLC plate 200 is the same as the structure of TLC plate 100 in the first exemplary embodiment except for functional layer 30. Therefore, constituent elements which are common between TLC plate 100 in the first exemplary embodiment and TLC plate 200 in the present exemplary embodiment are denoted by the same reference marks and may not be described in detail below. That is, the descriptions regarding the following exemplary embodiments are mutually applicable, in so far as they are technically consistent with one another. In addition, the respective exemplary embodiments may be combined with one another, in so far as they are technically consistent with one another.

Functional layer 30 has a rectangular shape in a plan view. Functional layer 30 extends in second development direction Y. Functional layer 30 is in contact with first layer 31. When separation layer 21 is viewed in a plan view, one side (long side) of first layer 31 is in contact with one side of functional layer 30. The length of one side of functional layer 30 is equal to the length of one side (long side) of first layer 31. First layer 31 and functional layer 30 constitute boundary face 41. Boundary face 41 extends in first development direction X. Functional layer 30 extends from an end face of substrate 10 in second development direction Y to boundary face 41. Functional layer 30, first layer 31, and second layer 32 are arrayed in this order in second development direction Y.

In the present exemplary embodiment, first layer 31, second layer 32, and functional layer 30 are disposed on substrate 10. In other words, first layer 31, second layer 32, and functional layer 30 are in contact with substrate 10. A lateral surface of first layer 31 and a lateral surface of second layer 32 are in contact with each other. A lateral surface of first layer 31 and a lateral surface of functional layer 30 are in contact with each other. When the multiple components are developed in second development direction Y, the developing solvent can easily move from functional layer 30 to first layer 31 through boundary face 41.

Functional layer 30 includes a hydrophobic porous body. The hydrophobic porous body may be the same as any of those described as examples of the porous body constituting second layer 32. An average pore diameter of functional layer 30 may be within a range from 0.01 μm to 100 μm. When the hydrophobic porous body is an aggregate of inorganic particles modified with a hydrophobic functional group, an average particle diameter of the inorganic particles may be within a range from 1 μm to 100 μm. Functional layer 30 may further include any of the additives mentioned above.

A composition of functional layer 30 may be the same as or different from a composition of second layer 32. A structure of functional layer 30 may be the same as or different from a structure of second layer 32. “The structure of functional layer 30 being different from the structure of second layer 32” means that at least one factor selected from among an average pore diameter of the porous body constituting functional layer 30, a void ratio of the porous body, and an average particle diameter of the material of the porous body is different from that of the porous body constituting second layer 32, for example.

Length L6 of functional layer 30 of TLC plate 200 in second development direction Y is not particularly limited. Length L6 is determined according to the porous body constituting functional layer 30, a size of a container for housing TLC plate 200, and the like. Length L6 is 5 mm to 50 mm, for example.

As a method for forming functional layer 30 on substrate 10, the methods described above as examples of the method for forming second layer 32 on substrate 10 in the first exemplary embodiment can be used, for example.

Each of second layer 32 and functional layer 30 of TLC plate 200 includes a hydrophobic porous body. Therefore, first developing solvent 70 is difficult to penetrate into each of second layer 32 and functional layer 30. That is, pores in the porous bodies constituting second layer 32 and functional layer 30 hardly contain first developing solvent 70, after the multiple components included in sample 60 are developed by first developing solvent 70. Therefore, it is unnecessary to dry second layer 32 and functional layer 30 after the multiple components included in the sample are developed in first development direction X.

According to TLC plate 200, the multiple components can move straight in second development direction Y in second layer 32. Specifically, when the multiple components are developed in second development direction Y, an end of functional layer 30 in second development direction Y is brought into contact with second developing solvent 71. In this case, a gradient may occur in a movement distance of second developing solvent 71 in second development direction Y. When the multiple components are developed with the gradient occurring in the movement distance, the multiple components may move in second layer 32 in a direction different from second development direction Y. However, when the gradient occurs in the movement distance, a portion of second developing solvent 71 moves in first development direction X as well as in second development direction Y. Therefore, the gradient in the movement distance of second developing solvent 71 is reduced, as second developing solvent 71 moves in functional layer 30. In TLC plate 200, when second developing solvent 71 moves from functional layer 30 to first layer 31, the gradient in the movement distance of second developing solvent 71 in second development direction Y is reduced. Thus, the multiple components can move straight in second development direction Y in second layer 32.

### Modification of Second Exemplary Embodiment

As shown in FIG. 3C, first layer 31 may be disposed on second layer 32 and functional layer 30. In TLC plate 210, second layer 32 and functional layer 30 are disposed on substrate 10. Second layer 32 is not in contact with functional layer 30. Space 50 is formed between second layer 32 and functional layer 30. First layer 31 is in contact with second layer 32 and functional layer 30. A lower surface of first layer 31 and an upper surface of second layer 32 constitute boundary face 42. The lower surface of first layer 31 and an upper surface of functional layer 30 constitute boundary face 43. Boundary faces 42 and 43 extend in first development direction X. When the sample is developed in second development direction Y, second developing solvent 71 moves from functional layer 30 to first layer 31 through boundary face 43. Second developing solvent 71 moves from first layer 31 to second layer 32 through boundary face 42. Due to space 50, second developing solvent 71 does not directly move to second layer 32 from functional layer 30. Therefore, the multiple components located in first layer 31 can easily move to second layer 32.

TLC plate 210 can be manufactured in such a way that second layer 32 and functional layer 30 are formed on substrate 10, and then, first layer 31 is formed on second layer 32 and functional layer 30. As a method for forming first layer 31 on second layer 32 and functional layer 30, the methods described above as examples of the method for forming first layer 31 on substrate 10 in the first exemplary embodiment can be used, for example. In TLC plate 210, first layer 31 is formed after the formation of second layer 32 and functional layer 30, whereby separation layer 21 can be easily manufactured.

### Another Modification of Second Exemplary Embodiment

As shown in FIG. 3D, second layer 32 may be in contact with functional layer 30. In TLC plate 220, a lateral surface of second layer 32 and a lateral surface of functional layer 30 constitute boundary face 44. First layer 31 is disposed on second layer 32 and functional layer 30. A lower surface of first layer 31 and upper surfaces of second layer 32 and functional layer 30 constitute boundary face 45. Boundary faces 44 and 45 extend in first development direction X. When the sample is developed in second development direction Y, the developing solvent moves from functional layer 30 to second layer 32 through boundary face 44.

TLC plate 220 is manufactured in the same manner as TLC plate 210. In TLC plate 220, first layer 31 is formed after the formation of second layer 32 and functional layer 30, whereby separation layer 21 can be easily manufactured.

In TLC plate 220, first developing solvent 70 is difficult to penetrate into each of second layer 32 and functional layer 30. Therefore, when being developed by first developing solvent 70, the multiple components included in sample 60 are held in first layer 31. Then, TLC plate 220 is brought into contact with second developing solvent 71. At that time, second layer 32 and functional layer 30 are both in contact with second developing solvent 71. Therefore, the porous bodies constituting second layer 32 and functional layer 30 get wet with second developing solvent 71. In this case, the multiple components held in first layer 31 tend to move to second layer 32 or functional layer 30 through boundary face 45. That is, when the porous bodies constituting second layer 32 and functional layer 30 get wet, the multiple components tend to move in the thickness direction of separation layer 21. This tendency is significant when alcohol is used as the organic solvent contained in second developing solvent 71. Due to the movement of the multiple components to second layer 32 or functional layer 30, the multiple components can be developed in second development direction Y.

### Third Exemplary Embodiment

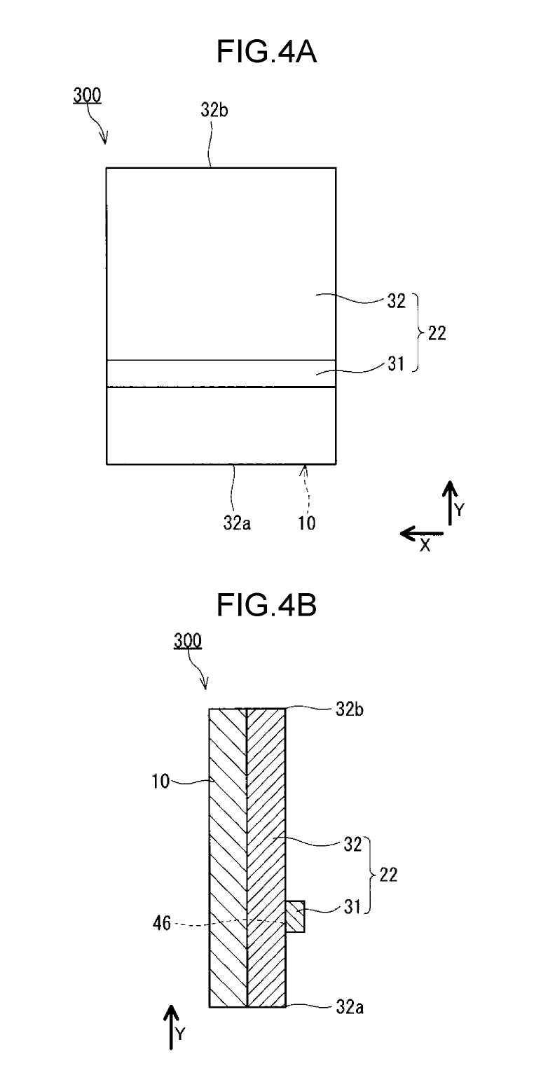

As shown in FIGS. 4A and 4B, in TLC plate 300 according to the present third exemplary embodiment, first layer 31 is disposed on second layer 32. Second layer 32 is disposed on substrate 10. In other words, only second layer 32 is in contact with substrate 10. Second layer 32 extends from one of a pair of end faces of substrate 10 to the other in second development direction Y. A lower surface of first layer 31 and an upper surface of second layer 32 are in contact with each other. First layer 31 and second layer 32 constitute boundary face 46. Boundary face 46 extends in first development direction X. First layer 31 is located between one end 32a and other end 32b of second layer 32 in second development direction Y.

A distance from one end 32a of second layer 32 to first layer 31 in second development direction Y is equal to a value that can be assumed by length L6 of functional layer 30 in TLC plate 200. A distance from first layer 31 to other end 32b of second layer 32 in second development direction Y is equal to a value that can be assumed by length L3 of second layer 32 in TLC plate 100.

TLC plate 300 can be manufactured in such a way that second layer 32 is formed on substrate 10, and then, first layer 31 is formed on second layer 32. As a method for forming second layer 32 on substrate 10 and a method for forming first layer 31 on second layer 32, the methods exemplified in the first exemplary embodiment can be used, for example. In TLC plate 300, first layer 31 is formed after the formation of second layer 32, whereby separation layer 22 can be easily manufactured.

In TLC plate 300, first developing solvent 70 is difficult to penetrate into second layer 32. Therefore, when being developed by first developing solvent 70, the multiple components included in sample 60 are held in first layer 31. Then, TLC plate 300 is brought into contact with second developing solvent 71. At that time, second layer 32 is in contact with second developing solvent 71. Thus, the porous body constituting second layer 32 gets wet with second developing solvent 71. In this case, the multiple components held in first layer 31 tend to move to second layer 32 through boundary face 46. That is, when the porous body constituting second layer 32 gets wet, the multiple components tend to move in the thickness direction of separation layer 22.

This tendency is significant when alcohol is used as the organic solvent contained in second developing solvent 71. Due to the movement of the multiple components to second layer 32, the multiple components can be developed in second development direction Y.

According to TLC plate 300, the multiple components can move straight in second development direction Y in second layer 32. Specifically, when the multiple components are developed in second development direction Y, second developing solvent 71 is brought into contact with one end 32a of second layer 32. In this case, a gradient may occur in a movement distance of second developing solvent 71 in second development direction Y. When the multiple components are developed with the gradient occurring in the movement distance, the multiple components may move in second layer 32 in a direction different from second development direction Y. However, when the gradient occurs in the movement distance, a portion of second developing solvent 71 moves in first development direction X as well as in second development direction Y. Therefore, the gradient in the movement distance of second developing solvent 71 is reduced, as second developing solvent 71 moves in second layer 32. In TLC plate 300, when second developing solvent 71 moves from one end 32a of second layer 32 to first layer 31, the gradient in the movement distance of second developing solvent 71 in second development direction Y is reduced. Thus, the multiple components can move straight in second development direction Y in second layer 32.

### Fourth Exemplary Embodiment

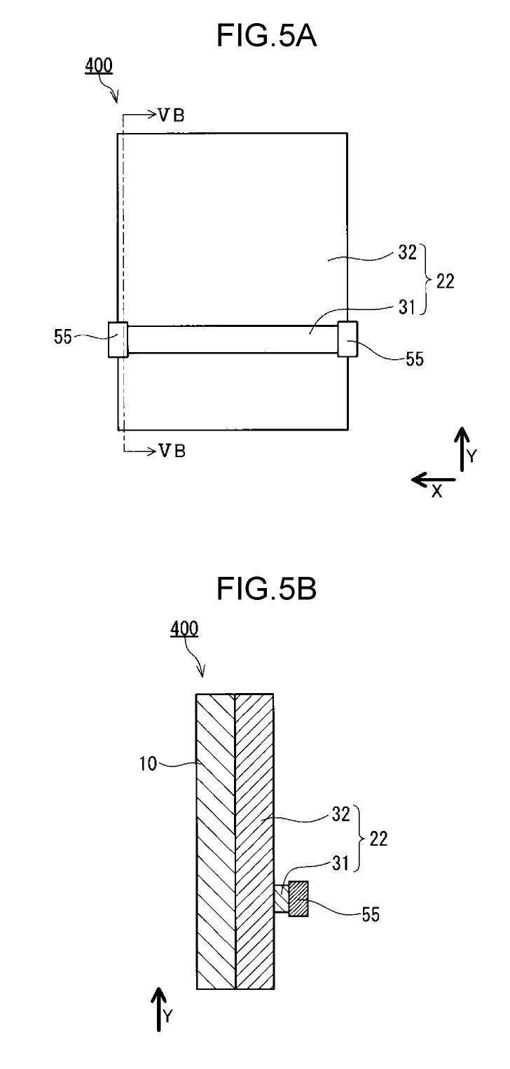

The TLC plate may further include a pair of electrodes. In FIGS. 5A and 5B, TLC plate 400 has a pair of electrodes 55. The pair of electrodes 55 is disposed at both ends of first layer 31 in first development direction X. The pair of electrodes 55 is disposed on first layer 31. If a voltage is applied to the pair of electrodes 55 with first layer 31 being impregnated with first developing solvent 70, current flows through first layer 31. A structure of TLC plate 400 is the same as the structure of TLC plate 300 in the third exemplary embodiment except for the pair of electrodes 55. Note that an average pore diameter of the porous material constituting first layer 31 of TLC plate 400 may be within a range from 0.1 μm to 100 μm. With this configuration, the multiple components included in the sample can be easily electrophoresed in first layer 31.

The pair of electrodes 55 is not particularly limited, as long as they can apply a voltage. The pair of electrodes 55 may be formed from at least one metal selected from the group consisting of platinum, gold, copper, and aluminum, for example.

Next, a sample analysis method using TLC plate 400 will be described.

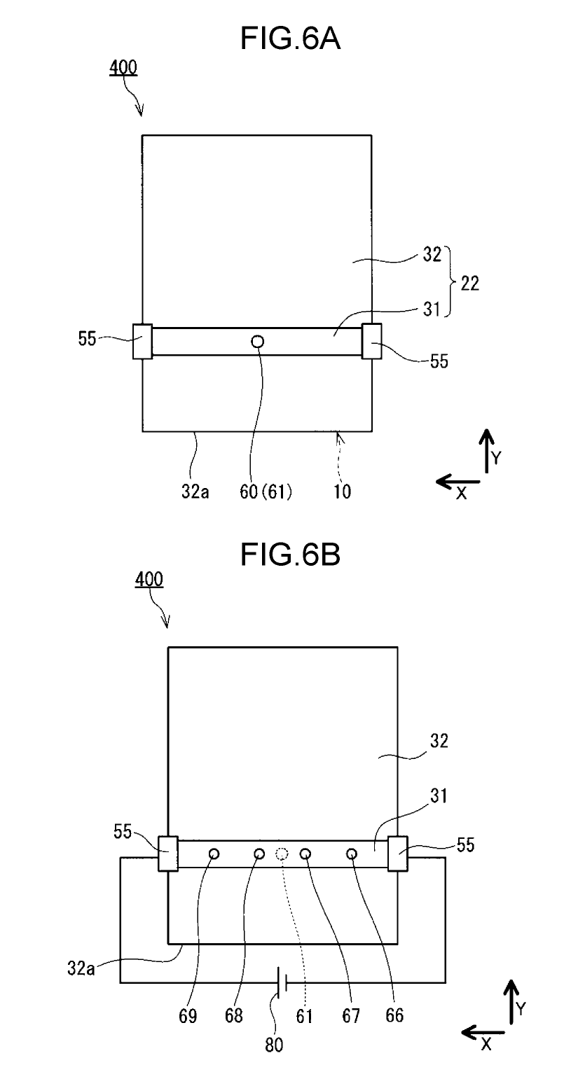

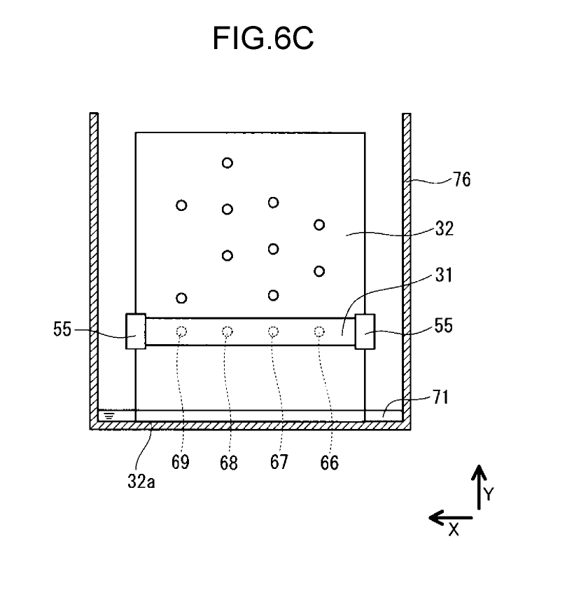

First, sample 60 is placed on first layer 31 of separation layer 22 of TLC plate 400, as shown in FIG. 6A. When sample 60 is placed on first layer 31, sample 60 penetrates into first layer 31, so that circular spot 61 is formed. Sample 60 is an aqueous solution containing a plurality of proteins, for example. In first layer 31, a position where sample 60 is to be placed is not particularly limited. Sample 60 may be placed on a middle point of first layer 31 in first development direction X. In this case, the multiple components included in sample 60 can be quickly separated from each other by electrophoresis of the multiple components. First layer 31 is impregnated with first developing solvent 70 in advance. First developing solvent 70 is typically the same as that used in the first exemplary embodiment.

Next, a voltage is applied to electrodes 55 on TLC plate 400 as shown in FIG. 6B. The multiple components included in sample 60 are electrophoresed in first development direction X. The voltage can be applied by power source 80. Power source 80 is an AC-to-DC converter, a power generating device, or a battery, for example. The electrophoresis may be conducted inside an analyzing device. The multiple components are separated from each other in first layer 31 based on isoelectric point or molecular weight of each component. Due to the electrophoresis of the multiple components in first development direction X, spots 66, 67, 68, and 69 are newly generated.

Then, as shown in FIG. 6C, TLC plate 400 is placed in container 76 with end 32a of second layer 32 in second development direction Y being directed downward. Container 76 contains second developing solvent 71. Container 76 and second developing solvent 71 are typically the same as those used in the first exemplary embodiment.

When TLC plate 400 is placed in container 76, end 32a of second layer 32 is in contact with second developing solvent 71. At that time, first layer 31 is impregnated with first developing solvent 70. The liquid level of second developing solvent 71 is set to prevent direct contact between second developing solvent 71 and spots 66, 67, 68, and 69. The porous body constituting second layer 32 gets wet with second developing solvent 71 by second developing solvent 71. Thus, the multiple components move from first layer 31 to second layer 32. Due to capillary force, second developing solvent 71 moves in second development direction Y from end 32a of second layer 32. When second developing solvent 71 is brought into contact with the multiple components located in spots 66, 67, 68, and 69, the multiple components are dissolved into second developing solvent 71. The multiple components dissolved in second developing solvent 71 move in second development direction Y along with second developing solvent 71. The multiple components which are not separated from each other in first layer 31 are separated from each other in second layer 32.

Second layer 32 of TLC plate 400 includes a hydrophobic porous body. First developing solvent 70 hardly penetrates into second layer 32. That is, pores in the porous body constituting second layer 32 hardly contain first developing solvent 70 after the electrophoresis of the multiple components included in sample 60. Therefore, it is unnecessary to dry second layer 32 after the multiple components included in the sample are developed in first development direction X. TLC plate 400 can be brought into contact with second developing solvent 71 with first layer 31 being impregnated with first developing solvent 70. In other words, it is unnecessary to heat TLC plate 400 to a temperature higher than room temperature or leave TLC plate 400 in an atmosphere with a pressure lower than atmospheric pressure, during a period from when the voltage applied to electrodes 55 is removed till TLC plate 400 is brought into contact with second developing solvent 71. According to TLC plate 400, the multiple components can be developed in second development direction Y just after the development of the multiple components in first development direction X. Thus, the multiple components can be separated from each other more easily and more quickly. Note that, with the sample analysis method in the present exemplary embodiment, separation layer 22 may be dried before TLC plate 400 is brought into contact with second developing solvent 71.

With the sample analysis method according to the present exemplary embodiment, it is unnecessary to change the orientation of TLC plate 400. Therefore, the analyzing device used for the sample analysis method according to the present exemplary embodiment does not need a mechanism for changing the orientation of the TLC plate.

As a method for detecting the positions of the multiple components and a method for identifying each of the multiple components in the sample analysis method according to the present exemplary embodiment, the methods described in the first exemplary embodiment can be used.

### Fifth Exemplary Embodiment

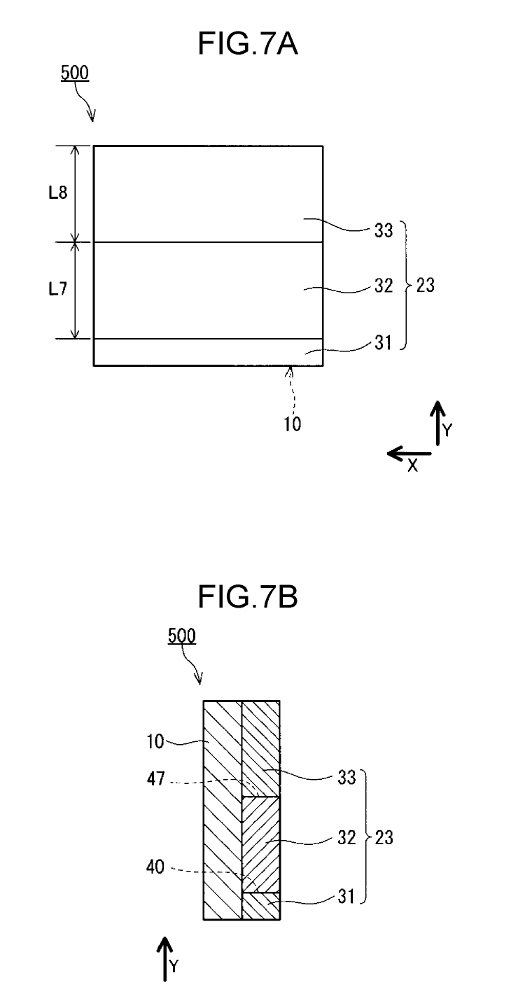

As shown in FIGS. 7A and 7B, TLC plate 500 according to the present fifth exemplary embodiment is obtained by further providing third layer 33 to the configuration of TLC plate 100 in the first exemplary embodiment. Third layer 33 induces an interaction different from the interaction induced by second layer 32, with respect to multiple components included in a sample. Therefore, the multiple components which are not separated from each other in second layer 32 are separated from each other in third layer 33.

Third layer 33 has a rectangular shape in a plan view. Third layer 33 extends in second development direction Y. Third layer 33 is in contact with second layer 32. When separation layer 23 is viewed in a plan view, one side of second layer 32 is in contact with one side of third layer 33. The length of one side of third layer 33 is equal to the length of one side of second layer 32. Third layer 33 and second layer 32 constitute boundary face 47. Boundary face 47 extends in first development direction X. Third layer 33 extends from boundary face 47 to an end face of substrate 10 in second development direction Y. First layer 31, second layer 32, and third layer 33 are arrayed in this order in second development direction Y.

In the present exemplary embodiment, first layer 31, second layer 32, and third layer 33 are disposed on substrate 10. In other words, first layer 31, second layer 32, and third layer 33 are in contact with substrate 10. A lateral surface of first layer 31 and a lateral surface of second layer 32 are in contact with each other. A lateral surface of second layer 32 and a lateral surface of third layer 33 are in contact with each other. When the multiple components are developed in second development direction Y, a developing solvent can easily move from second layer 32 to third layer 33 through boundary face 47.

Third layer 33 includes a porous body. The porous body constituting third layer 33 may be the same as any of those described as examples of the porous body constituting first layer 31 or second layer 32. When the multiple components included in the sample are developed in first development direction X by electrophoresis, third layer 33 may includes a hydrophilic porous body. An average pore diameter of third layer 33 may be within a range from 0.01 μm to 100 μm. When the porous body is an aggregate of inorganic particles, an average particle diameter of the inorganic particles may be within a range from 1 μm to 100 μm. Third layer 33 may further include any of the additives mentioned above.

TLC plate 500 satisfies at least one requirement selected from among a requirement in which a composition of third layer 33 is different from a composition of second layer 32 and a requirement in which a structure of third layer 33 is different from a structure of second layer 32. Thus, third layer 33 induces an interaction different from the interaction induced by second layer 32, with respect to the multiple components included in the sample. “The structure of third layer 33 being different from the structure of second layer 32” means that at least one factor selected from among an average pore diameter of the porous body constituting third layer 33, a void ratio of the porous body, and an average particle diameter of the material of the porous body is different from that of the porous body constituting second layer 32, for example. The multiple components which are not separated from each other in second layer 32 are separated from each other in third layer 33.

Length L7 of second layer 32 in second development direction Y is not particularly limited. Length L7 is determined according to the porous body constituting second layer 32, a size of a container for housing TLC plate 500, and the like.

Length L8 of third layer 33 in second development direction Y is not particularly limited. Length L8 is determined according to the porous body constituting third layer 33, a size of a container for housing TLC plate 500, and the like.

As a method for forming third layer 33 on substrate 10, the methods described above as examples of the method for forming first layer 31 on substrate 10 and the method for forming second layer 32 on substrate 10 in the first exemplary embodiment can be used, for example.

TLC plate 500 may further include functional layer 30 provided to TLC plate 200 in the second exemplary embodiment. In this configuration, functional layer 30, first layer 31, second layer 32, and third layer 33 are arrayed in this order in second development direction Y.

### Sixth Exemplary Embodiment

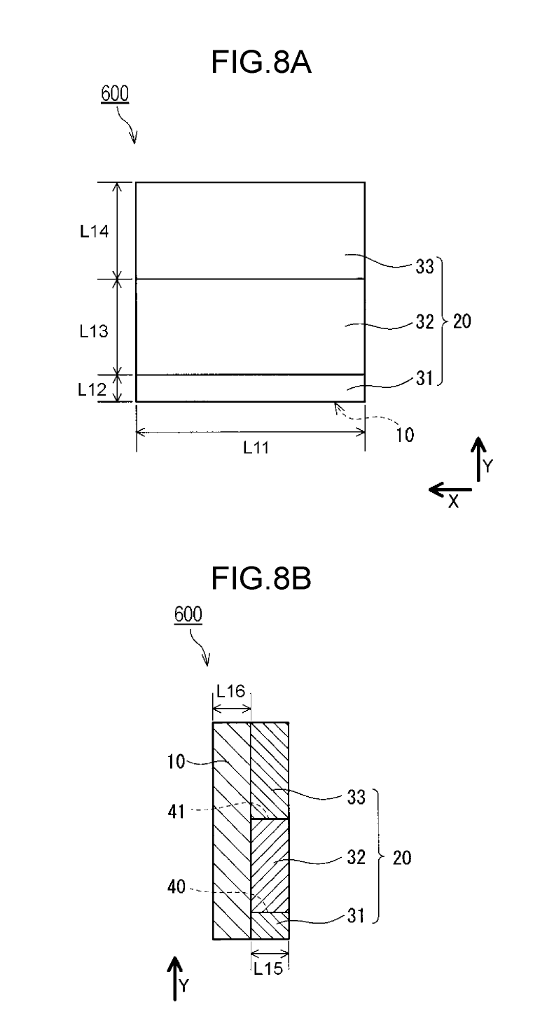

As shown in FIGS. 8A and 8B, thin layer chromatography plate 600 (hereinafter referred to as “TLC plate 600”) according to the present first exemplary embodiment has substrate 10 and separation layer 20. Substrate 10 has a plate shape, for example. Substrate 10 has, for example, a rectangular shape in a plan view. Separation layer 20 is disposed on substrate 10. Separation layer 20 covers the surface of substrate 10. Substrate 10 has two pairs of end faces facing each other. In the present exemplary embodiment, first development direction X extends from one end face of one of two pairs of end faces of substrate 10 to the other end face. Multiple components included in the sample are developed in first development direction X in a first stage. Second development direction Y extends from one end face of the other of two pairs of end faces of substrate 10 to the other end face. Multiple components included in the sample are developed in second development direction Y in a second stage. Second development direction Y is orthogonal to first development direction X.

Separation layer 20 separates the multiple components included in the sample from each other. Separation layer 20 includes first layer 31, second layer 32, and third layer 33. First layer 31 is a band-shaped layer. First layer 31 has a rectangular band shape in a plan view. First layer 31 extends in first development direction X. First layer 31 extends from one of a pair of end faces of substrate 10 to the other in first development direction X. Note that first layer 31 may not extend to the other end face of substrate 10.

Second layer 32 has a rectangular shape in a plan view. Second layer 32 extends in second development direction Y. Second layer 32 is in contact with first layer 31. When separation layer 20 is viewed in a plan view, one side (long side) of first layer 31 is in contact with one side of second layer 32.

The length of one side (long side) of first layer 31 is equal to the length of one side of second layer 32. First layer 31 and second layer 32 constitute boundary face 40. Boundary face 40 extends in first development direction X. Second layer 32 extends to third layer 33 from boundary face 40.

Third layer 33 has a rectangular shape in a plan view. Third layer 33 extends in second development direction Y. Third layer 33 is in contact with second layer 32. When separation layer 20 is viewed in a plan view, one side of second layer 32 is in contact with one side of third layer 33. The length of one side of third layer 33 is equal to the length of one side of second layer 32. Third layer 33 and second layer 32 constitute boundary face 41. Boundary face 41 extends in first development direction X. Third layer 33 extends from boundary face 41 to an end face of substrate 10 in second development direction Y. Note that third layer 33 may not extend to the end face of substrate 10. First layer 31, second layer 32, and third layer 33 are arrayed in this order in second development direction Y.

In the present exemplary embodiment, first layer 31, second layer 32, and third layer 33 are disposed on substrate 10. In other words, first layer 31, second layer 32, and third layer 33 are in contact with substrate 10. A lateral surface of first layer 31 and a lateral surface of second layer 32 are in contact with each other. A lateral surface of second layer 32 and a lateral surface of third layer 33 are in contact with each other. When the multiple components are developed in second development direction Y, a developing solvent can easily move from first layer 31 to second layer 32 through boundary face 40. The developing solvent can easily move from second layer 32 to third layer 33 through boundary face 41.

The material of substrate 10 is not particularly limited, as long as it can maintain the shape of TLC plate 600 without being eluted in the developing solvent. The material of substrate 10 is glass, resin, metal, or paper, for example. Substrate 10 is typically a glass plate or an aluminum film.

First layer 31 includes a hydrophilic porous body. In the present specification, “being hydrophilic” means that, when water is brought into contact with the porous body, water can move in the porous body at a rate of 5 mm/min or higher due to capillary force, for example. When water is selected as the developing solvent, first layer 31 can carry water from one end to the other end of first layer 31 in first development direction X due to capillary force. The hydrophilic porous body is not particularly limited. The material of the hydrophilic porous body includes at least one selected from the group consisting of a fiber material, and an inorganic material and a polymer material which are hydrophilic, for example.

The fiber material includes at least one selected from the group consisting of a plant fiber, an animal fiber, a recycled fiber, a synthetic fiber, and a glass fiber, for example. The plant fiber includes cellulose, for example. The synthetic fiber includes cellulose acetate, for example.

The hydrophilic polymer material includes at least one selected from the group consisting of agarose, dextran, and mannan, for example. The inorganic material includes at least one selected from the group consisting of alumina, silicon dioxide, and zirconia, for example.

The hydrophilic porous body is filter paper, for example. The hydrophilic porous body is an aggregate of at least one kind of inorganic particles selected from the group consisting of alumina particles, silica gel particles, silicon pillar, zeolite particles, diatom earth, and zirconia particles, for example.

An average pore diameter of first layer 31 may be within a range from 0.01 μm to 100 μm. When the hydrophilic porous body is an aggregate of inorganic particles, an average particle diameter of the inorganic particles may be within a range from 1 μm to 100 μm. The “average pore diameter” can be measured with the following method. Specifically, the surface or cross-section of first layer 31 is observed with an electron microscope (for example, an electron scanning microscope). Pore diameters of a plurality of observed pores (for example, random 50 pores) are measured. The average pore diameter is determined based on the average value calculated using the measured values. The diameter of a circle having an area equal to the area of the pore observed with the electron microscope can be regarded as the pore diameter. The “average particle diameter” can be measured with the following method. Specifically, the surface or cross-section of first layer 31 is observed with an electron microscope, and diameters of random number (for example, 50) of particles constituting first layer 31 are measured. The average particle diameter is determined based on the average value calculated using the obtained measured values. The diameter of a circle having an area equal to the area of the particle observed with the electron microscope can be regarded as the particle diameter.

First layer 31 may further include an additive. Examples of the additive include a fluorescence indicator, a binder, and a metal oxide.

Examples of the fluorescence indicator include magnesium tungstate and zinc silicate containing manganese. When first layer 31 includes the fluorescence indicator, positions of the multiple components can be detected by irradiating first layer 31 with ultraviolet ray.

The binder includes at least one selected from the group consisting of an inorganic binder, an organic fiber, a thickener, and an organic binder, for example. Examples of the inorganic binder include plaster and colloidal silica. Examples of the organic fiber include microfibrillar cellulose. Examples of the thickener include hydroxyethyl cellulose and carboxymethyl cellulose. Examples of the organic binder include polyvinyl alcohol and polyacrylic acid. When first layer 31 includes the binder, adhesiveness between substrate 10 and first layer 31 is improved. When the porous body of first layer 31 is an aggregate of inorganic particles, durability of the aggregate of inorganic particles is improved due to the binder.

The metal oxide includes at least one selected from the group consisting of titanium oxide, aluminum oxide, tin oxide, zinc oxide, tungsten oxide, manganese oxide, nickel oxide, copper oxide, and magnesium oxide, for example. The metal oxide may be charged when getting wet with the developing solvent. Therefore, when first layer 31 includes the metal oxide, the interaction between the multiple components included in the sample and first layer 31 varies. Thus, the multiple components may be easily separated from each other in first layer 31.

The above-mentioned additives may be mixed into the porous body constituting first layer 31. When the additive is a binder or a metal oxide, the additive may cover the surface of the porous body. The additive may coat the surfaces of inorganic particles constituting the porous body.

Second layer 32 includes a hydrophobic porous body. In the present specification, “being hydrophobic” means that, when water is brought into contact with the porous body, water can move in the porous body at a rate lower than 5 mm/min due to capillary force, or water does not move inside the porous body due to capillary force, for example. When water moves inside the hydrophobic porous body, the movement rate of water may be lower than 1 mm/min. When second layer 32 is brought into contact with water, water hardly penetrates into second layer 32. When the developing solvent contains an organic solvent, second layer 32 can carry the developing solvent from one end to the other end of second layer 32 in second development direction Y due to capillary force. The hydrophobic porous body is not particularly limited.

The material of the hydrophobic porous body is, for example, a hydrophobic polymer material. The hydrophobic polymer material includes at least one selected from the group consisting of fluororesin, polystyrene, polyethylene, and polypropylene, for example.

The hydrophobic porous body is, for example, a porous body of a hydrophobic polymer material or an aggregate of hydrophobic polymer material particles. The hydrophobic porous body is an aggregate of inorganic particles modified with a hydrophobic functional group, for example. The hydrophobic functional group includes a functional group having a hydrocarbon group at the end, for example. The hydrocarbon group includes at least one selected from the group consisting of an octadecyl group, an octyl group, a t-butyl group, a trimethylsilyl group, and a phenyl group, for example. The inorganic particles include at least one kind selected from the group consisting of alumina particles, silica gel particles, silicon pillar, zeolite particles, diatom earth, and zirconia particles, for example. The hydrophobic porous body is typically an aggregate of silica gel particles modified with a hydrophobic functional group. Whether the inorganic particles are modified with a hydrophobic functional group can be confirmed by conducting an element analysis on the cross-section of second layer 32, for example. The element analysis can be conducted by X-ray photoelectron spectroscopy (XPS) or energy dispersive X-ray spectroscopy (EDX), for example.

An average pore diameter of second layer 32 may be within a range from 0.01 μm to 100 μm. When the hydrophobic porous body is an aggregate of inorganic particles modified with a hydrophobic functional group, an average particle diameter of the inorganic particles may be within a range from 1 μm to 100 μm. Second layer 32 may further include any of the above-mentioned additives.

Third layer 33 includes a porous body. The porous body constituting third layer 33 may be the same as any of those described as examples of the porous body constituting first layer 31 or second layer 32. When the multiple components included in the sample are developed in first development direction X by electrophoresis, third layer 33 may include a hydrophilic porous body. An average pore diameter of third layer 33 may be within a range from 0.01 μm to 100 μm. When the porous body is an aggregate of inorganic particles, an average particle diameter of the inorganic particles may be within a range from 1 μm to 100 μm. Third layer 33 may further include any of the additives mentioned above.

TLC plate 600 satisfies at least one requirement selected from among a requirement in which a composition of third layer 33 is different from a composition of second layer 32 and a requirement in which a structure of third layer 33 is different from a structure of second layer 32. Thus, third layer 33 induces an interaction different from the interaction induced by second layer 32, with respect to the multiple components included in the sample. “The structure of third layer 33 being different from the structure of second layer 32” means that at least one factor selected from among an average pore diameter of the porous body constituting third layer 33, a void ratio of the porous body, and an average particle diameter of the material of the porous body is different from that of the porous body constituting second layer 32, for example.

Length L11 of first layer 31 in first development direction X is not particularly limited. Length L11 is determined according to the porous body constituting first layer 31, a size of a container for housing TLC plate 600, and the like. Length L11 is 10 mm to 100 mm, for example. The length of second layer 32 and the length of substrate 10 in first development direction X are typically equal to length L11.

Length L12 of first layer 31 in second development direction Y is not particularly limited. Length L12 is determined according to an amount of the sample to be placed on first layer 31, for example. As length L12 is smaller, the multiple components are more easily separated from each other when the multiple components are developed in second development direction Y. Length L12 is 0.5 mm to 10 mm, for example.

Length L13 of second layer 32 in second development direction Y is not particularly limited. Length L13 is determined according to the porous body constituting second layer 32, a size of a container for housing TLC plate 600, and the like.

Length L14 of third layer 33 in second development direction Y is not particularly limited. Length L14 is determined according to the porous body constituting third layer 33, a size of a container for housing TLC plate 600, and the like. The length of substrate 10 in second development direction Y is typically equal to the total of length L12, length L13, and length L14.

Thickness L15 of first layer 31 is not particularly limited. Thickness L15 is determined according to the porous body constituting first layer 31. Thickness L15 is 0.05 mm to 1 mm, for example. The thickness of second layer 32 and the thickness of third layer 33 are typically equal to thickness L15 of first layer 31.

Thickness L16 of substrate 10 is not particularly limited as long as the shape of TLC plate 600 can be maintained. Thickness L16 is 0.1 mm to 5 mm, for example.

Next, a manufacturing method of TLC plate 100 will be described.

First, a first dispersion liquid containing inorganic particles is prepared. The first dispersion liquid can be obtained by dispersing inorganic particles into a coating solvent.

The coating solvent includes at least one selected from the group consisting of water and an organic solvent, for example. The organic solvent includes at least one selected from the group consisting of alcohol, ketone, ether, nitrile, sulfoxide, sulfone, ester, carboxylic acid, amide, hydrocarbon, aromatic hydrocarbon, and halogen-containing compound, for example. Examples of alcohol include methanol, ethanol, and isopropyl alcohol. Examples of ketone include acetone and ethyl methyl ketone. Examples of ether include tetrahydrofuran and dioxane. Examples of nitrile include acetonitrile. Examples of sulfoxide include dimethyl sulfoxide. Examples of sulfone include sulfolane. Examples of ester include ethyl acetate. Examples of carboxylic acid includes formic acid and acetic acid. Examples of amide include dimethylformamide. Examples of hydrocarbon include pentane and hexane. Examples of aromatic hydrocarbon include benzene, toluene, and xylene. Examples of halogen-containing compound include methylene chloride, chloroform, bromoform, chlorobenzene, and bromobenzene.

The first dispersion liquid is applied on a part of the surface of substrate 10 to form a coating film. The coating film is dried, whereby first layer 31 is formed on substrate 10. When the hydrophilic porous body is filter paper, first layer 31 is formed on substrate 10 by bonding the hydrophilic porous body to a part of the surface of substrate 10 under pressure.

Next, a second dispersion liquid containing inorganic particles modified with a hydrophobic functional group is prepared. The second dispersion liquid can be obtained by dispersing inorganic particles modified with a hydrophobic functional group into a coating solvent. The materials mentioned above can be used for the coating solvent.

The second dispersion liquid is applied on a part of the surface of substrate 10 to form a coating film. The coating film is dried, whereby second layer 32 is formed on substrate 10. When the hydrophobic porous body is a porous body of a hydrophobic polymer material, second layer 32 is formed on substrate 10 by bonding the hydrophobic porous body to a part of the surface of substrate 10 under pressure.

The second dispersion liquid may contain inorganic particles not modified with a hydrophobic functional group, in place of inorganic particles modified with a hydrophobic functional group. In such a case, second layer 32 is formed in the manner described below. The second dispersion liquid is applied on substrate 10 to form a coating film. The coating film is dried, whereby a untreated layer of second layer 32 is formed. A silane coupling agent having a hydrophobic functional group is applied on the untreated layer. The silane coupling agent is reacted with the inorganic particles included in the untreated layer. Thus, second layer 32 is formed on substrate 10. The silane coupling agent may be applied on a coating film, not on the untreated layer.

The silane coupling agent is not particularly limited. The silane coupling agent may include at least one selected from the group consisting of dimethyloctadecylchlorosilane, dimethyloctylchlorosilane, t-butyldimethylchlorosilane, trimethylchlorosilane, and phenyldimethylchlorosilane.

First layer 31 and second layer 32 may be formed by the following method. The first dispersion liquid is applied on the entire surface of substrate 10 to form a coating film The coating film is dried, whereby a untreated layer of second layer 32 and first layer 31 are formed on substrate 10. A silane coupling agent having a hydrophobic functional group is applied on the untreated layer of second layer 32. The silane coupling agent is reacted with the inorganic particles. Thus, first layer 31 and second layer 32 are formed on substrate 10.

Next, third layer 33 is formed on substrate 10. As a method for forming third layer 33 on substrate 10, the methods described above as examples of the method for forming first layer 31 on substrate 10 and the method for forming second layer 32 on substrate 10 can be used, for example.

The order of formation of first layer 31, second layer 32, and third layer 33 on substrate 10 is not particularly limited. First layer 31 and second layer 32 may be respectively formed on substrate 10 after third layer 33 is formed on substrate 10.

Next, a sample analysis method using TLC plate 600 will be described.

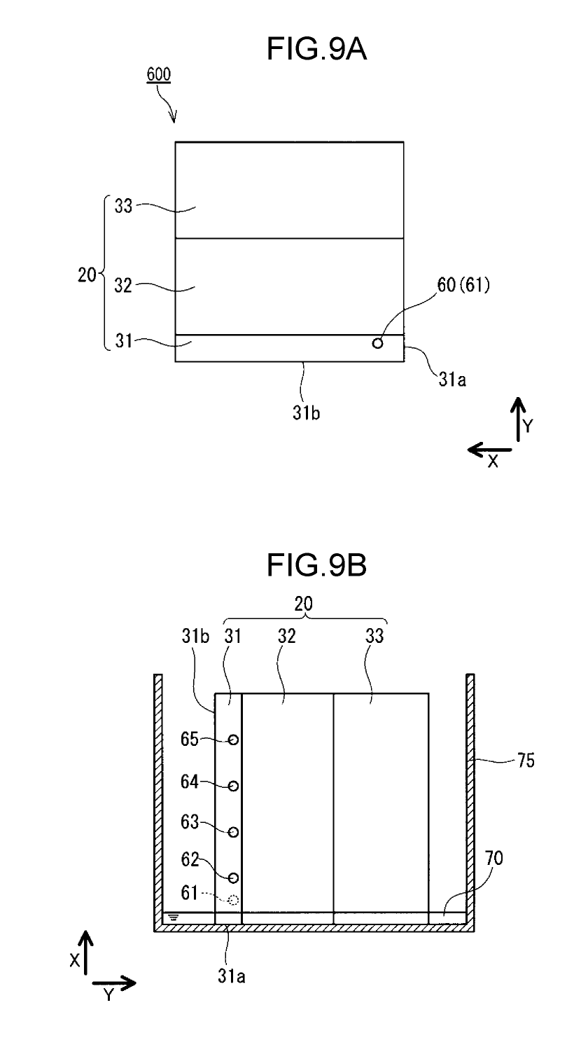

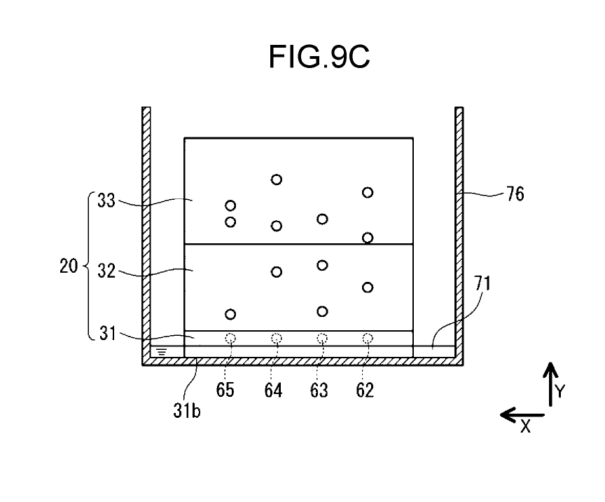

First, sample 60 is placed on first layer 31 of separation layer 20 of TLC plate 600, as shown in FIG. 9A. When sample 60 is placed on first layer 31, sample 60 penetrates into first layer 31, so that circular spot 61 is formed. Sample 60 is an aqueous solution containing a plurality of proteins, for example. The content of the plurality of proteins in sample 60 is from 0.01 wt. % to 1 wt. %, for example. The volume of sample 60 placed on first layer 31 is 0.1 μL to 2 μL, for example. The position where sample 60 is to be placed on first layer 31 is not particularly limited, as long as sample 60 is not in direct contact with the first developing solvent and the second developing solvent.

Then, as shown in FIG. 9B, TLC plate 600 is placed in container 75 with end 31a of first layer 31 in first development direction X being directed downward. Container 75 contains first developing solvent 70. Container 75 is a glass jar, for example. Container 75 may be installed inside an analyzing device (not shown).

First developing solvent 70 is not particularly limited, as long as it does not move to the inside of second layer 32 or third layer 33 when being in contact with the surface of second layer 32 or third layer 33. In the sample analysis method according to the present exemplary embodiment, third layer 33 can include a hydrophobic porous body. First developing solvent 70 is water or an aqueous solution, for example. A solute of the aqueous solution contains at least one selected from the group consisting of phosphate, citrate, acetate, and borate, for example. The aqueous solution may be a buffer solution such as a phosphate buffer solution, a tris buffer solution, a citrate buffer solution, an acetate buffer solution, or a borate buffer solution. In the present exemplary embodiment, first developing solvent 70 does not contain an organic solvent. However, first developing solvent 70 may contain an organic solvent. When first developing solvent 70 contains an organic solvent, first developing solvent 70 typically contains 80 volume % or more water.

When TLC plate 600 is placed in container 75, end 31a of first layer 31 is in contact with first developing solvent 70. The liquid level of first developing solvent 70 is set to prevent direct contact between first developing solvent 70 and sample 60. Due to the capillary force, first developing solvent 70 moves in first development direction X from end 31a of first layer 31. When first developing solvent 70 and sample 60 are brought into contact with each other, the multiple components included in sample 60 are dissolved into first developing solvent 70. The multiple components dissolved in first developing solvent 70 move in first development direction X along with first developing solvent 70. The multiple components move while repeatedly adsorbing and desorbing to and from the porous body constituting first layer 31. The frequency of adsorption and desorption varies in each component, and thus, the multiple components are separated from each other in first layer 31. Due to the development of sample 60 in first development direction X, spots 62, 63, 64, and 65 are newly generated. Spots 62, 63, 64, and 65 respectively indicate that any of the multiple components included in the sample is located therein.

Then, the orientation of TLC plate 600 is changed. The analyzing device may include a mechanism for changing the orientation of TLC plate 600. As shown in FIG. 9C, TLC plate 600 is placed in container 76 with end 31b of first layer 31 in second development direction Y being directed downward. Container 76 contains second developing solvent 71. Container 76 is a glass jar, for example. Container 76 may be installed inside the analyzing device.

Second developing solvent 71 is not particularly limited, as long as it contains an organic solvent. Second developing solvent 71 contains an organic solvent, so that it can penetrate into second layer 32 and third layer 33. The materials mentioned above as examples of the coating solvent can be used as the organic solvent. The organic solvent contains at least one selected from the group consisting of methanol, ethanol, isopropyl alcohol, acetonitrile, and acetic acid, for example. When second developing solvent 71 contains carboxylic acid and the sample contains proteins, the frequency of absorption and desorption of proteins to and from the porous bodies constituting second layer 32 and third layer 33 is improved. Second developing solvent 71 may contain the organic solvent in an amount of 20 wt. % or more. Second developing solvent 71 may contain water in addition to the organic solvent. That is, second developing solvent 71 may be a mixed solvent containing the organic solvent and water. When second developing solvent 71 contains water and the sample contains proteins, solubility of the proteins in second developing solvent 71 is improved. In other words, the multiple components included in the sample can be easily dissolved in second developing solvent 71. Specific examples of second developing solvent 71 include a mixed solvent in which isopropyl alcohol, acetic acid, and water are mixed in a weight ratio of 40:5:55.

When TLC plate 600 is placed in container 76, end 31b of first layer 31 is in contact with second developing solvent 71. At that time, first layer 31 is impregnated with first developing solvent 70. The liquid level of second developing solvent 71 is set to prevent direct contact between second developing solvent 71 and spots 62, 63, 64, and 65. Second developing solvent 71 penetrates into first layer 31. Second developing solvent 71 moves from end 31b of first layer 31 in second development direction Y due to capillary force, along with first developing solvent 70 penetrating into first layer 31. When second developing solvent 71 is brought into contact with the multiple components located in spots 62, 63, 64, and 65, the multiple components are dissolved into second developing solvent 71. The multiple components dissolved in second developing solvent 71 move in second development direction Y along with second developing solvent 71. The multiple components move while repeatedly adsorbing and desorbing to and from the porous body constituting second layer 32 or third layer 33. The multiple components which are not separated from each other in first layer 31 are separated from each other in second layer 32. The multiple components which are not separated from each other in second layer 32 are separated from each other in third layer 33.

Second layer 32 of TLC plate 600 includes a hydrophobic porous body. First developing solvent 70 hardly penetrates into second layer 32. That is, pores in the porous body constituting second layer 32 hardly contain first developing solvent 70, after the multiple components included in sample 60 are developed by first developing solvent 70. Therefore, it is unnecessary to dry second layer 32 after the multiple components included in the sample are developed in first development direction X. TLC plate 100 can be brought into contact with second developing solvent 71 with first layer 31 being impregnated with first developing solvent 70. In other words, it is unnecessary to heat TLC plate 600 to a temperature higher than room temperature or leave TLC plate 600 in an atmosphere with a pressure lower than atmospheric pressure, during a period from when TLC plate 600 is lifted up from first developing solvent 70 till TLC plate 600 is brought into contact with second developing solvent 71. According to TLC plate 600, the multiple components can be developed in second development direction Y just after the development of the multiple components in first development direction X. Thus, the multiple components can be separated from each other more easily and more quickly. Note that, with the sample analysis method in the present exemplary embodiment, separation layer 20 may be dried before TLC plate 600 is brought into contact with second developing solvent 71.

A method for detecting positions of multiple components is not particularly limited, and any known methods can be employed. For example, when each of first layer 31, second layer 32, and third layer 33 contains a fluorescence indicator, separation layer 20 may be irradiated with ultraviolet ray to detect the positions of multiple components. In such a case, each of the multiple components can be a compound that absorbs ultraviolet ray. The analyzing device may have a mechanism for emitting ultraviolet ray. The positions of the multiple components may be detected by depositing a coloring reagent onto separation layer 20. In such a case, TLC plate 600 may be heated as necessary. Any known coloring reagent can be used. Examples of the coloring reagent include anisaldehyde, phosphomolybdic acid, iodine, ninhydrin, chameleon solution, 2,4-dinitrophenylhydrazine, manganese chloride, and bromocresol green.

Under the same condition, the positions of the multiple components after sample 60 is developed are determined for each component. Therefore, with the sample analysis method according to the present exemplary embodiment, each of the separated multiple components can be identified. For example, a component having a known structure is developed on TLC plate 600 under the condition same as the condition for developing sample 60. Data in which the position of the component after the development and the structure of the component are associated with each other is acquired. This data may be stored in a memory of the analyzing device in advance. Through comparison with the data, each of the multiple components can be identified based on the position of each component after sample 60 is developed.

### Seventh Exemplary Embodiment

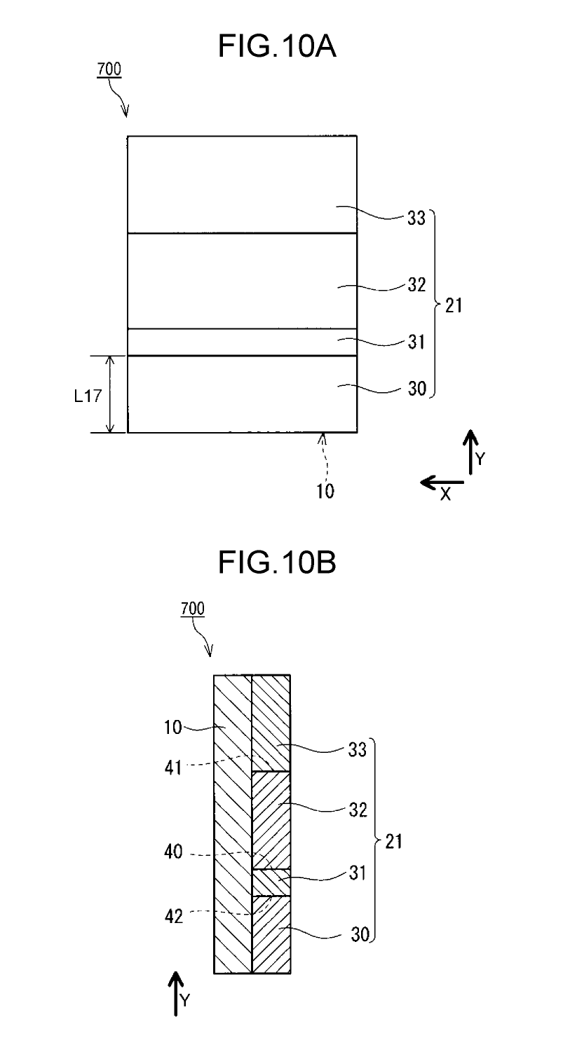

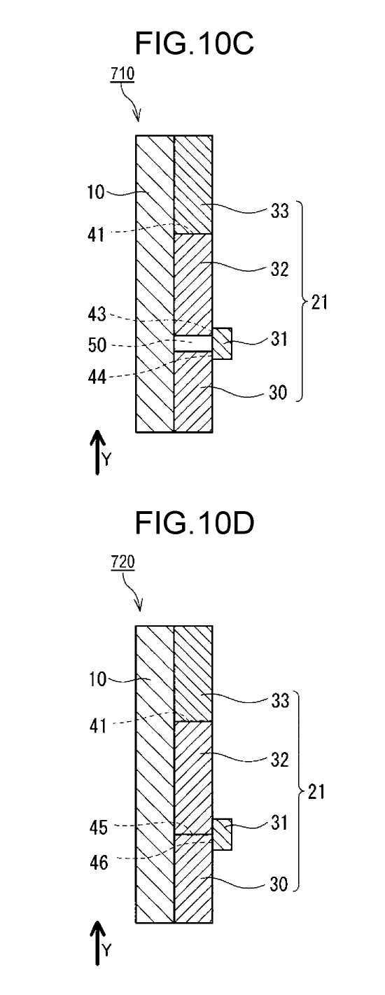

As shown in FIGS. 10A and 10B, TLC plate 700 according to the present seventh exemplary embodiment includes separation layer 21 having first layer 31, second layer 32, third layer 33, and functional layer 30. A structure of TLC plate 700 is the same as the structure of TLC plate 600 in the sixth exemplary embodiment except for functional layer 30. Therefore, constituent elements which are common between TLC plate 700 in the first exemplary embodiment and TLC plate 700 in the present exemplary embodiment are denoted by the same reference marks and may not be described in detail below. That is, the descriptions regarding the following exemplary embodiments are mutually applicable, in so far as they are technically consistent with one another. In addition, the respective exemplary embodiments may be combined with one another, in so far as they are technically consistent with one another.

Functional layer 30 has a rectangular shape in a plan view. Functional layer 30 extends in second development direction Y. Functional layer 30 is in contact with first layer 31. When separation layer 21 is viewed in a plan view, one side (long side) of first layer 31 is in contact with one side of functional layer 30. The length of one side of functional layer 30 is equal to the length of one side (long side) of first layer 31. First layer 31 and functional layer 30 constitute boundary face 42. Boundary face 42 extends in first development direction X. Functional layer 30 extends from an end face of substrate 10 in second development direction Y to boundary face 42. Functional layer 30, first layer 31, second layer 32, and third layer 33 are arrayed in this order in second development direction Y.

In the present exemplary embodiment, first layer 31, second layer 32, third layer 33, and functional layer 30 are disposed on substrate 10. In other words, first layer 31, second layer 32, third layer 33, and functional layer 30 are in contact with substrate 10. A lateral surface of first layer 31 and a lateral surface of second layer 32 are in contact with each other. A lateral surface of second layer 32 and a lateral surface of third layer 33 are in contact with each other. A lateral surface of first layer 31 and a lateral surface of functional layer 30 are in contact with each other. When the multiple components are developed in second development direction Y, a developing solvent can easily move from functional layer 30 to first layer 31 through boundary face 42.

Functional layer 30 includes a hydrophobic porous body. The hydrophobic porous body may be the same as any of those described as examples of the porous body constituting second layer 32. An average pore diameter of functional layer 30 may be within a range from 0.01 μm to 100 μm. When the hydrophobic porous body is an aggregate of inorganic particles modified with a hydrophobic functional group, an average particle diameter of the inorganic particles may be within a range from 1 μm to 100 μm. Functional layer 30 may further include any of the additives mentioned above.

A composition of functional layer 30 may be the same as or different from a composition of second layer 32. A structure of functional layer 30 may be the same as or different from a structure of second layer 32. “The structure of functional layer 30 being different from the structure of second layer 32” means that at least one factor selected from among an average pore diameter of the porous body constituting functional layer 30, a void ratio of the porous body, and an average particle diameter of the material of the porous body is different from that of the porous body constituting second layer 32, for example.

Length L17 of functional layer 30 in second development direction Y is not particularly limited in TLC plate 700. Length L17 is determined according to the porous body constituting functional layer 30, a size of a container for housing TLC plate 700, and the like. Length L17 is 5 mm to 50 mm, for example.

As a method for forming functional layer 30 on substrate 10, the methods described above as examples of the method for forming second layer 32 on substrate 10 in the sixth exemplary embodiment can be used, for example.

Each of second layer 32 and functional layer 30 of TLC plate 700 includes a hydrophobic porous body. Therefore, first developing solvent 70 is difficult to penetrate into each of second layer 32 and functional layer 30. That is, pores in the porous bodies constituting second layer 32 and functional layer 30 hardly contain first developing solvent 70, after the multiple components included in sample 60 are developed by first developing solvent 70. Therefore, it is unnecessary to dry second layer 32 and functional layer 30 after the multiple components included in the sample are developed in first development direction X.

According to TLC plate 700, the multiple components can move straight in second development direction Y in second layer 32 and third layer 33. Specifically, when the multiple components are developed in second development direction Y, an end of functional layer 30 in second development direction Y is brought into contact with second developing solvent 71. In this case, a gradient may occur in a movement distance of second developing solvent 71 in second development direction Y. When the multiple components are developed with the gradient occurring in the movement distance, the multiple components may move in second layer 32 and third layer 33 in a direction different from second development direction Y. However, when the gradient occurs in the movement distance, a portion of second developing solvent 71 moves in first development direction X as well as in second development direction Y. Therefore, the gradient in the movement distance of second developing solvent 71 is reduced, as second developing solvent 71 moves in functional layer 30. In TLC plate 700, when second developing solvent 71 moves from functional layer 30 to first layer 31, the gradient in the movement distance of second developing solvent 71 in second development direction Y is reduced. Thus, the multiple components can move straight in second development direction Y in second layer 32 and third layer 33.

### Modification of Seventh Exemplary Embodiment

As shown in FIG. 10C, first layer 31 may be disposed on second layer 32 and functional layer 30. In TLC plate 710, second layer 32, third layer 33, and functional layer 30 are disposed on substrate 10. Second layer 32 is not in contact with functional layer 30. Space 50 is formed between second layer 32 and functional layer 30. First layer 31 is in contact with second layer 32 and functional layer 30. A lower surface of first layer 31 and an upper surface of second layer 32 constitute boundary face 43. The lower surface of first layer 31 and an upper surface of functional layer 30 constitute boundary face 44. Boundary faces 43 and 44 extend in first development direction X. First layer 31 is not in contact with third layer 33. When the sample is developed in second development direction Y, second developing solvent 71 moves from functional layer 30 to first layer 31 through boundary face 44. Second developing solvent 71 moves from first layer 31 to second layer 32 through boundary face 43. Due to space 50, second developing solvent 71 does not directly move to second layer 32 from functional layer 30. Therefore, the multiple components located in first layer 31 can easily move to second layer 32.

TLC plate 710 can be manufactured in such a way that second layer 32 and functional layer 30 are formed on substrate 10, and then, first layer 31 is formed on second layer 32 and functional layer 30. As a method for forming first layer 31 on second layer 32 and functional layer 30, the methods described above as examples of the method for forming first layer 31 on substrate 10 in the first exemplary embodiment can be used, for example. In TLC plate 710, first layer 31 is formed after the formation of second layer 32, third layer 33, and functional layer 30, whereby separation layer 21 can be easily manufactured.

### Another Modification of Second Exemplary Embodiment

As shown in FIG. 10D, second layer 32 may be in contact with functional layer 30. In TLC plate 720, a lateral surface of second layer 32 and a lateral surface of functional layer 30 constitute boundary face 45. First layer 31 is disposed on second layer 32 and functional layer 30. A lower surface of first layer 31 and upper surfaces of second layer 32 and functional layer 30 constitute boundary face 46. First layer 31 is not in contact with third layer 33. Boundary faces 45 and 46 extend in first development direction X. When the sample is developed in second development direction Y, the developing solvent moves from functional layer 30 to second layer 32 through boundary face 45.

TLC plate 720 is manufactured in the same manner as TLC plate 710. In TLC plate 720, first layer 31 is formed after the formation of second layer 32, third layer 33, and functional layer 30, whereby separation layer 21 can be easily manufactured.

In TLC plate 720, first developing solvent 70 is difficult to penetrate into each of second layer 32 and functional layer 30. Therefore, when being developed by first developing solvent 70, the multiple components included in sample 60 are held in first layer 31. Then, TLC plate 720 is brought into contact with second developing solvent 71. At that time, second layer 32 and functional layer 30 are both in contact with second developing solvent 71. Therefore, the porous bodies constituting second layer 32 and functional layer 30 get wet with second developing solvent 71. In this case, the multiple components held in first layer 31 tend to move to second layer 32 or functional layer 30 through boundary face 46. That is, when the porous bodies constituting second layer 32 and functional layer 30 get wet, the multiple components tend to move in the thickness direction of separation layer 21. This tendency is significant when alcohol is used as the organic solvent contained in second developing solvent 71. Due to the movement of the multiple components to second layer 32 or functional layer 30, the multiple components can be developed in second development direction Y.

### Eighth Exemplary Embodiment

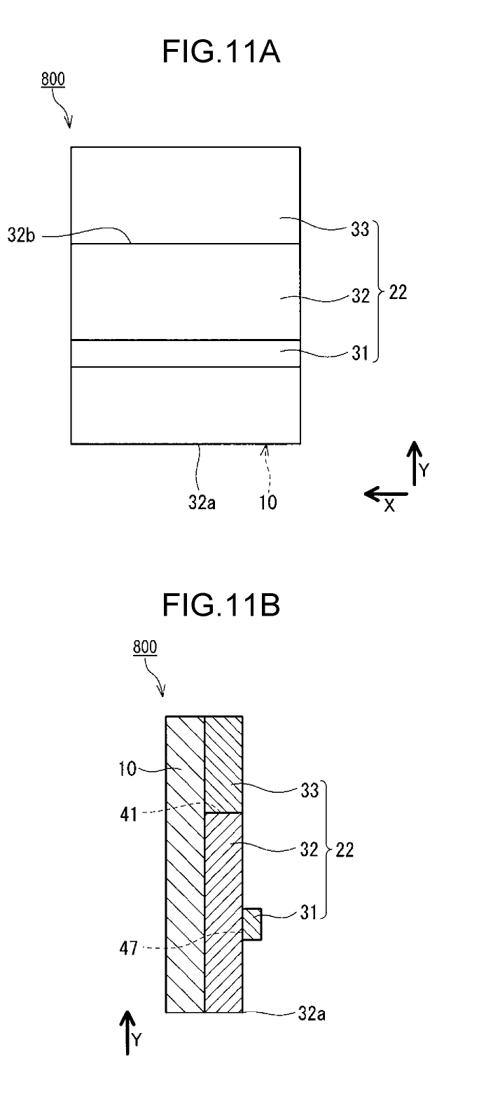

As shown in FIGS. 11A and 11B, in TLC plate 800 according to the present third exemplary embodiment, first layer 31 is disposed on second layer 32. Second layer 32 and third layer 33 are disposed on substrate 10. In other words, only second layer 32 and third layer 33 are in contact with substrate 10. Second layer 32 extends from an end face of substrate 10 in second development direction Y to boundary face 41 between second layer 32 and third layer 33. A lower surface of first layer 31 and an upper surface of second layer 32 are in contact with each other. First layer 31 and second layer 32 constitute boundary face 47. Boundary face 47 extends in first development direction X. First layer 31 is located between one end 32a and other end 32b of second layer 32 in second development direction Y. First layer 31 is not in contact with third layer 33.

A distance from one end 32a of second layer 32 to first layer 31 in second development direction Y is equal to a value that can be assumed by length L17 of functional layer 30 in TLC plate 700. A distance from first layer 31 to other end 32b of second layer 32 in second development direction Y is equal to a value that can be assumed by length L13 of second layer 32 in TLC plate 600.

TLC plate 800 can be manufactured in such a way that second layer 32 is formed on substrate 10, and then, first layer 31 is formed on second layer 32. As a method for forming second layer 32 on substrate 10 and a method for forming first layer 31 on second layer 32, the methods exemplified in the sixth exemplary embodiment can be used, for example. In TLC plate 800, first layer 31 is formed after the formation of second layer 32, whereby separation layer 22 can be easily manufactured.

In TLC plate 800, first developing solvent 70 is difficult to penetrate into second layer 32. Therefore, when being developed by first developing solvent 70, the multiple components included in sample 60 are held in first layer 31. Then, TLC plate 800 is brought into contact with second developing solvent 71. At that time, second layer 32 is in contact with second developing solvent 71. Thus, the porous body constituting second layer 32 gets wet with second developing solvent 71. In this case, the multiple components held in first layer 31 tend to move to second layer 32 through boundary face 47. That is, when the porous body constituting second layer 32 gets wet, the multiple components tend to move in the thickness direction of separation layer 22. This tendency is significant when alcohol is used as the organic solvent contained in second developing solvent 71. Due to the movement of the multiple components to second layer 32, the multiple components can be developed in second development direction Y.

According to TLC plate 800, the multiple components can move straight in second development direction Y in second layer 32 and third layer 33. Specifically, when the multiple components are developed in second development direction Y, second developing solvent 71 is brought into contact with one end 32a of second layer 32. In this case, a gradient may occur in a movement distance of second developing solvent 71 in second development direction Y. When the multiple components are developed with the gradient occurring in the movement distance, the multiple components may move in second layer 32 and third layer 33 in a direction different from second development direction Y. However, when the gradient occurs in the movement distance, a portion of second developing solvent 71 moves in first development direction X as well as in second development direction Y. Therefore, the gradient in the movement distance of second developing solvent 71 is reduced, as second developing solvent 71 moves in second layer 32. In TLC plate 800, when second developing solvent 71 moves from one end 32a of second layer 32 to first layer 31, the gradient in the movement distance of second developing solvent 71 in second development direction Y is reduced. Thus, the multiple components can move straight in second development direction Y in second layer 32 and third layer 33.

### Ninth Exemplary Embodiment

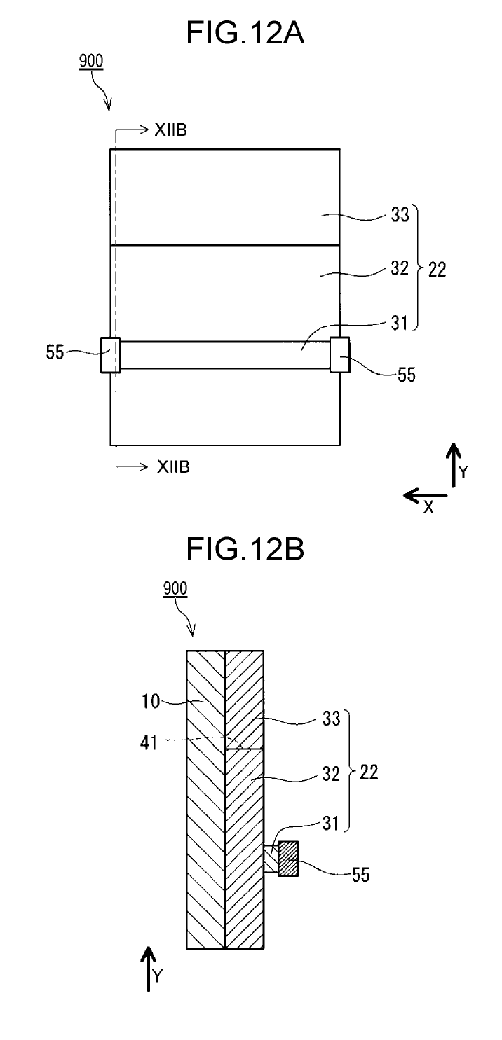

The TLC plate may further include a pair of electrodes. In FIGS. 12A and 12B, TLC plate 900 has a pair of electrodes 55. The pair of electrodes 55 is disposed at both ends of first layer 31 in first development direction X. The pair of electrodes 55 is disposed on first layer 31. If a voltage is applied to the pair of electrodes 55 with first layer 31 being impregnated with first developing solvent 70, current flows through first layer 31. A structure of TLC plate 900 is the same as the structure of TLC plate 800 in the eighth exemplary embodiment except for the pair of electrodes 55. Note that an average pore diameter of the porous material constituting first layer 31 of TLC plate 900 may be within a range from 0.1 μm to 100 μm. With this configuration, the multiple components included in the sample can be easily electrophoresed in first layer 31.

The pair of electrodes 55 is not particularly limited, as long as they can apply a voltage. The pair of electrodes 55 may be formed from at least one metal selected from the group consisting of platinum, gold, copper, and aluminum, for example.

Next, a sample analysis method using TLC plate 900 will be described.

First, sample 60 is placed on first layer 31 of separation layer 22 of

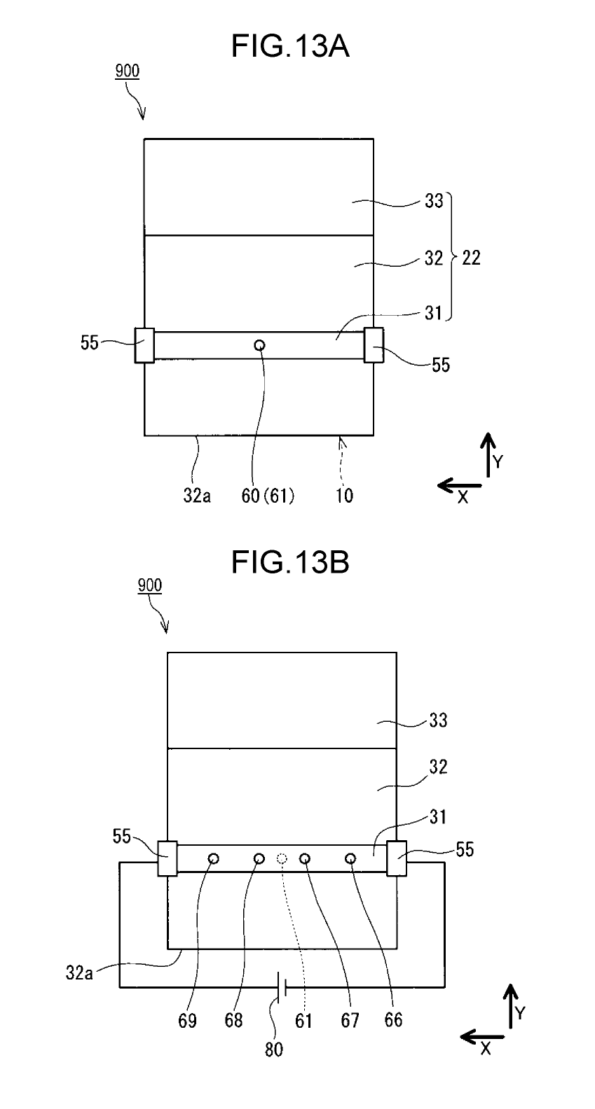

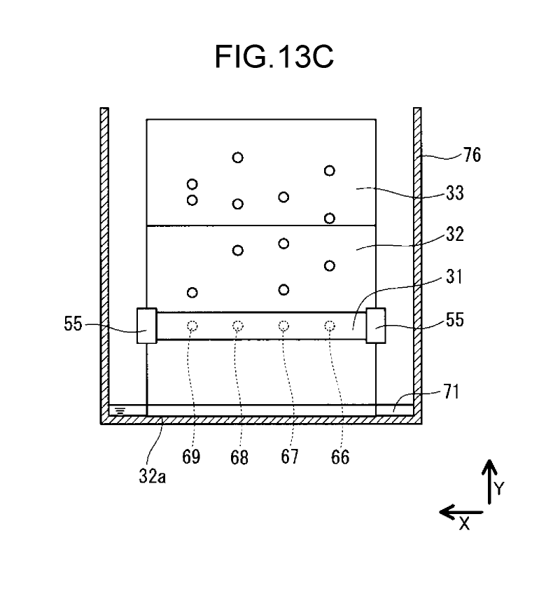

TLC plate 900, as shown in FIG. 13A. When sample 60 is placed on first layer 31, sample 60 penetrates into first layer 31, so that circular spot 61 is formed. Sample 60 is an aqueous solution containing a plurality of proteins, for example. In first layer 31, a position where sample 60 is to be placed is not particularly limited. Sample 60 may be placed on a middle point of first layer 31 in first development direction X. In this case, the multiple components included in sample 60 can be quickly separated from each other by electrophoresis of the multiple components. First layer 31 is impregnated with first developing solvent 70 in advance. First developing solvent 70 is typically the same as that used in the first exemplary embodiment.

Then, a voltage is applied to electrodes 55 on TLC plate 900 as shown in FIG. 13B. The multiple components included in sample 60 are electrophoresed in first development direction X. The voltage can be applied by power source 80. Power source 80 is an AC-to-DC converter, a power generating device, or a battery, for example. The electrophoresis may be conducted inside an analyzing device. The multiple components are separated from each other in first layer 31 based on isoelectric point or molecular weight of each component. Due to the electrophoresis of the multiple components in first development direction X, spots 66, 67, 68, and 69 are newly generated.

Then, as shown in FIG. 13C, TLC plate 900 is placed in container 76 with end 32a of second layer 32 in second development direction Y being directed downward. Container 76 contains second developing solvent 71. Container 76 and second developing solvent 71 are typically the same as those used in the sixth exemplary embodiment.

When TLC plate 900 is placed in container 76, end 32a of second layer 32 is in contact with second developing solvent 71. At that time, first layer 31 is impregnated with first developing solvent 70. The liquid level of second developing solvent 71 is set to prevent direct contact between second developing solvent 71 and spots 66, 67, 68, and 69. The porous body constituting second layer 32 gets wet with second developing solvent 71 by second developing solvent 71. Thus, the multiple components move from first layer 31 to second layer 32. Due to capillary force, second developing solvent 71 moves in second development direction Y from end 32a of second layer 32. When second developing solvent 71 is brought into contact with the multiple components located in spots 66, 67, 68, and 69, the multiple components are dissolved into second developing solvent 71. The multiple components dissolved in second developing solvent 71 move in second development direction Y along with second developing solvent 71. The multiple components which are not separated from each other in first layer 31 are separated from each other in second layer 32. The multiple components which are not separated from each other in second layer 32 are separated from each other in third layer 33.

Second layer 32 of TLC plate 900 includes a hydrophobic porous body. First developing solvent 70 hardly penetrates into second layer 32. That is, pores in the porous body constituting second layer 32 hardly contain first developing solvent 70 after the electrophoresis of the multiple components included in sample 60. Third layer 33 is not in contact with first layer 31. Therefore, first developing solvent 70 hardly penetrates into third layer 33. That is, pores in the porous body constituting third layer 33 hardly contain first developing solvent 70 after the electrophoresis of the multiple components included in sample 60. Therefore, it is unnecessary to dry second layer 32 and third layer 33 after the multiple components included in the sample are developed in first development direction X. TLC plate 900 can be brought into contact with second developing solvent 71 with first layer 31 being impregnated with first developing solvent 70. In other words, it is unnecessary to heat TLC plate 900 to a temperature higher than room temperature or leave TLC plate 900 in an atmosphere with a pressure lower than atmospheric pressure, during a period from when the voltage applied to electrodes 55 is removed till TLC plate 900 is brought into contact with second developing solvent 71. According to TLC plate 900, the multiple components can be developed in second development direction Y just after the development of the multiple components in first development direction X. Thus, the multiple components can be separated from each other more easily and more quickly. Note that, with the sample analysis method in the present exemplary embodiment, separation layer 22 may be dried before TLC plate 900 is brought into contact with second developing solvent 71.

With the sample analysis method according to the present exemplary embodiment, it is unnecessary to change the orientation of TLC plate 900. Therefore, the analyzing device used for the sample analysis method according to the present exemplary embodiment does not need a mechanism for changing the orientation of the TLC plate.

As a method for detecting the positions of the multiple components and a method for identifying each of the multiple components in the sample analysis method according to the present exemplary embodiment, the methods described in the first exemplary embodiment can be used.

### Tenth Exemplary Embodiment

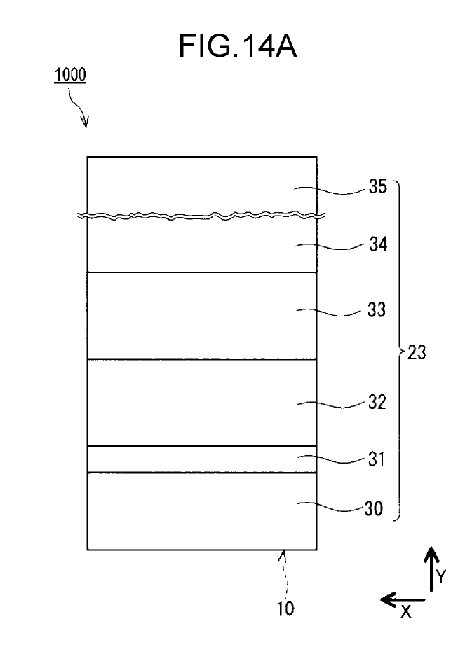

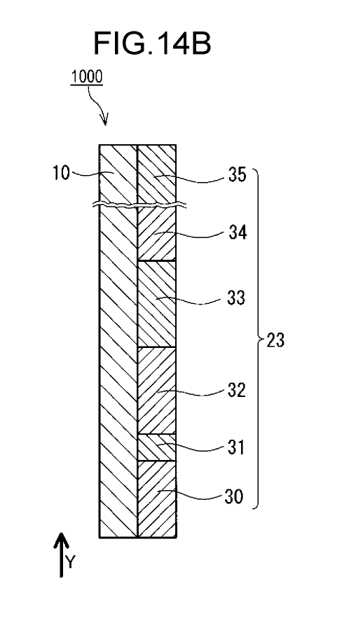

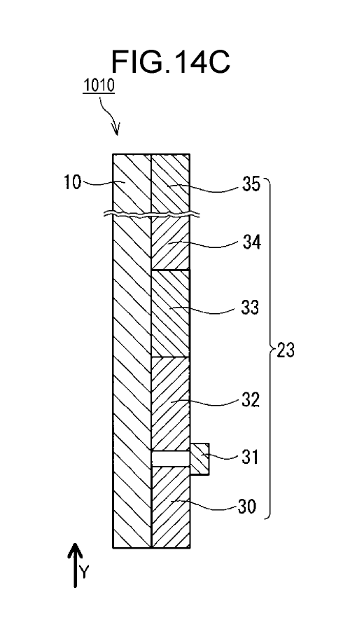

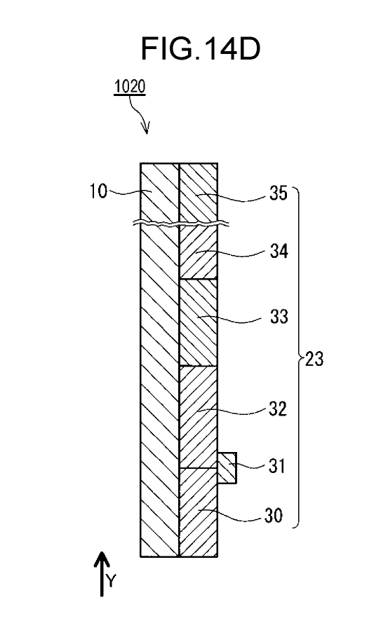

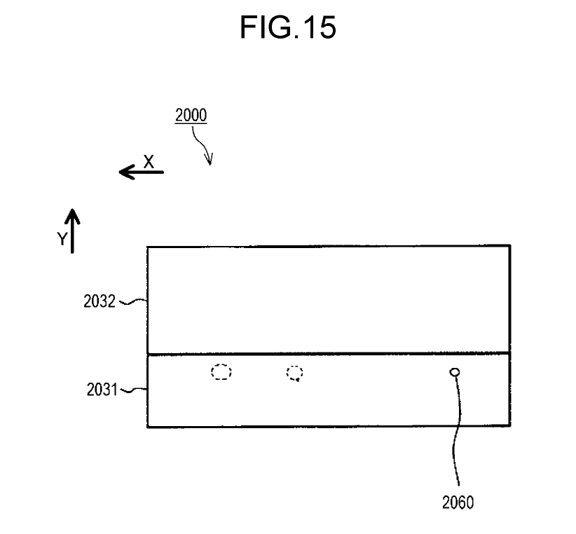

As shown in FIGS. 14A and 14B, TLC plate 1000 according to the present tenth exemplary embodiment is obtained by further providing fourth layer 34 to nth layer 35 to the configuration of TLC plate 700 in the second exemplary embodiment. Each of fourth layer 34 to nth layer 35 induces an interaction different from the interactions induced by second layer 32 and third layer 33, with respect to multiple components included in a sample. Therefore, the multiple components which are not separated from each other in second layer 32 and third layer 33 are separated from each other in fourth layer 34 to nth layer 35.

Each of fourth layer 34 to nth layer 35 has a rectangular shape in a plan view. Each of fourth layer 34 to nth layer 35 extends in second development direction Y. The n is an integer equal to or greater than 4. The n is an integer from 5 to 10, for example. Each of fourth layer 34 to nth layer 35 is in contact with the corresponding one of third layer 33 to (n-1)th layer (not shown). When separation layer 23 is viewed in a plan view, one side of each of fourth layer 34 to nth layer 35 is in contact with one side of the corresponding one of third layer 33 to (n-1)th layer. The length of one side of each of fourth layer 34 to nth layer 35 is equal to the length of one side of the corresponding one of third layer 33 to (n-1)th layer. Functional layer 30 and first layer 31 to nth layer 35 are arrayed in this order in second development direction Y.

In the present exemplary embodiment, functional layer 30 and first layer 31 to nth layer 35 are disposed on substrate 10. In other words, functional layer 30 and first layer 31 to nth layer 35 are in contact with substrate 10. A lateral surface of each of first layer 31 to nth layer 35 is in contact with a lateral surface of the corresponding one of functional layer 30 and first layer 31 to (n-1)th layer. When the multiple components are developed in second development direction Y, a developing solvent sequentially moves in the order of functional layer 30 and first layer 31 to nth layer 35.

Each of fourth layer 34 to nth layer 35 includes a porous body. The porous body constituting each of fourth layer 34 to nth layer 35 may be the same as any of those described as examples of the porous body constituting first layer 31 or second layer 32. When the multiple components included in the sample are developed in first development direction X by electrophoresis, each of fourth layer 34 to nth layer 35 may include a hydrophilic porous body. An average pore diameter of each of fourth layer 34 to nth layer 35 may be within a range from 0.01 μm to 100 μm. When the porous body is an aggregate of inorganic particles, an average particle diameter of the inorganic particles may be within a range from 1 μm to 100 μm. Fourth layer 34 to nth layer 35 may further include any of the additives mentioned above.

TLC plate 1000 satisfies at least one requirement selected from among a requirement in which second layer 32 to nth layer 35 are different in composition and a requirement in which second layer 32 to nth layer 35 are different in structure. Accordingly, second layer 32 to nth layer 35 induce different interactions with the multiple components included in the sample. “Second layer 32 to nth layer 35 being different in structure” means that second layer 32 to nth layer 35 are different in at least one factor selected from among an average pore diameter of the porous body, a void ratio of the porous body, and an average particle diameter of the material of the porous body, for example. The multiple components are separated from each other in each of second layer 32 to nth layer 35.

TLC plate 1000 may not satisfy the above-mentioned requirement depending on the multiple components included in sample 60. For example, at least two layers selected from second layer 32 to nth layer 35 may have the same composition and the same structure, as long as they are not in contact with each other.

Hydrophobic property may be improved in the order of second layer 32 to nth layer 35. Hydrophobic property may be decreased in the order of second layer 32 to nth layer 35. At least one factor selected from among the average pore diameter, the void ratio, and the average particle diameter of the material of the porous body constituting each of the second layer 32 to nth layer 35 may be increased in the order of second layer 32 to nth layer 35. At least one factor selected from among the average pore diameter, the void ratio, and the average particle diameter of the material of the porous body constituting each of the second layer 32 to nth layer 35 may be decreased in the order of second layer 32 to nth layer 35. The content of additives may be increased in the order of second layer 32 to nth layer 35. The content of additives may be decreased in the order of second layer 32 to nth layer 35.

The length of each of fourth layer 34 to nth layer 35 in second development direction Y is equal to a value that can be assumed by length L14 of third layer 33 of TLC plate 600. The lengths of second layer 32 to nth layer 35 in second development direction Y may be the same as or different from each other.

As a method for forming fourth layer 34 to nth layer 35 on substrate 10, the methods described above as examples of the method for forming first layer 31 on substrate 10 and the method for forming second layer 32 on substrate 10 in the first exemplary embodiment can be used, for example.

### Modification of Tenth Exemplary Embodiment

As shown in FIG. 14C, first layer 31 may be disposed on second layer 32 and functional layer 30. A structure of TLC plate 1010 is the same as the structure of TLC plate 710 in the seventh exemplary embodiment except for fourth layer 34 to nth layer 35.

### Another Modification of Tenth Exemplary Embodiment

As shown in FIG. 14D, second layer 32 may be in contact with functional layer 30. A structure of TLC plate 1020 is the same as the structure of TLC plate 720 in the seventh exemplary embodiment except for fourth layer 34 to nth layer 35.

### INDUSTRIAL APPLICABILITY

The technique disclosed in the present specification is useful for protein analysis or the like.

### REFERENCE MARKS IN THE DRAWINGS

10: substrate
20, 21, 22, 23: separation layer
30: functional layer
31: first layer
32: second layer
33: third layer
55: electrode
60: sample
91, 92, 93: end face
100, 200, 210, 220, 300, 400, 500, 600, 700, 710, 720, 800, 900, 1000,
1010, 1020: TLC plate (thin layer chromatography plate)
X: first development direction
y: second development direction

1. A thin layer chromatography plate comprising:
a substrate; and
a separation layer disposed on the substrate, the separation layer separating multiple components included in a sample from each other,
wherein
the separation layer has a first layer that has a band shape and extends in a first development direction and a second layer that extends in a second development direction orthogonal to the first development direction,
the second layer is in contact with the first layer,
the first layer includes a hydrophilic porous body, and
the second layer includes a hydrophobic porous body.

2. The thin layer chromatography plate according to claim 1, wherein
the first layer and the second layer are both disposed on the substrate, and
a lateral surface of the first layer and a lateral surface of the second layer are in contact with each other.

3. The thin layer chromatography plate according to claim 1, wherein
the separation layer further has a third layer in contact with the second layer,
the first layer, the second layer, and the third layer are arrayed in sequence in the second development direction,
the third layer includes a porous body, and
at least one requirement selected from among a requirement of a composition of the third layer being different from a composition of the second layer and a requirement of a structure of the third layer being different from a structure of the second layer is satisfied.

4. The thin layer chromatography plate according to claim 3, wherein
the first layer, the second layer, and the third layer are disposed on the substrate, and
the lateral surface of the first layer and the lateral surface of the second layer are in contact with each other.

5. The thin layer chromatography plate according to claim 2, wherein
the separation layer further includes a functional layer that extends in the second development direction and includes the hydrophobic porous body or another hydrophobic porous body,
the functional layer is in contact with the first layer, and
the functional layer, the first layer, and the second layer are arrayed in sequence in the second development direction.

6. The thin layer chromatography plate according to claim 1, wherein
the second layer is disposed on the substrate, the first layer is disposed on the second layer, and
a lower surface of the first layer and an upper surface of the second layer are in contact with each other.

7. The thin layer chromatography plate according to claim 6, wherein the first layer is located between one end and the other end of the second layer in the second development direction.

8. The thin layer chromatography plate according to claim 1, wherein the hydrophobic porous body is an aggregate of silica gel particles modified with a hydrophobic functional group.

9. The thin layer chromatography plate according to claim 1, further comprising a pair of electrodes disposed at both ends of the first layer in the first development direction.

10. A sample analysis method comprising:
placing a sample onto the first layer of the thin layer chromatography plate according to claim 1;
bringing an end of the first layer in the first development direction into contact with a first developing solvent; and
changing an orientation of the thin layer chromatography plate to bring the thin layer chromatography plate into contact with a second developing solvent containing an organic solvent with the first layer being impregnated with the first developing solvent.

11. The sample analysis method according to claim 10, wherein the first developing solvent is an aqueous solution.

12. The sample analysis method according to claim 10, wherein the second developing solvent is a mixed solvent containing the organic solvent and water.

13. A sample analysis method comprising:
placing a sample onto the first layer of the thin layer chromatography plate according to claim 9;
applying a voltage to the pair of electrodes; and
bringing the thin layer chromatography plate into contact with a developing solvent containing an organic solvent.
